(19) United States
(12) Patent Application Publication
(10) Pub. No.: US 2021/0338807 A1
(43) Pub. Date: Nov. 4, 2021

(54) COVID19 VACCINES AND RELATED METHODS

(71) Applicant: Humane Genomics, New York, NY (US)

(72) Inventor: Chad M. Moles, New York, NY (US)

(21) Appl. No.: 17/338,546

(22) Filed: Jun. 3, 2021

Related U.S. Application Data
(60) Provisional application No. 63/034,388, filed on Jun. 3, 2020.

(51) Int. Cl. A61K 39/215; C12N 7/00

(52) U.S. Cl. CPC A61K 39/215; A61K 2039/54; C12N 7/00

(57) ABSTRACT

The disclosure provides viral vaccine compositions for use in treating COVID-19 in a subject to whom the compositions are administered, as well as to methods of making and using the compositions.

### RELATED APPLICATIONS

This application claims the benefit of U.S. Provisional Application No. 63/034,388, filed on Jun. 3, 2020. The entire teachings of the above application are incorporated herein by reference.

### BACKGROUND OF THE INVENTION

Vesicular Stomatitis Virus (VSV) is a non-segmented negative-stranded RNA virus and belongs to the family Rhabdoviridae, genus Vesiculovirus. Its simple structure and rapid high-titered growth in mammalian and many other cell types has made it a preferential tool for molecular and cell biologists in the past 30 years. This was strengthened with the establishment of the reverse genetics system for VSV (Schnell et al., 1996).

VSV has been used as a viral backbone for vaccines for multiple pathogens and infectious diseases, including SARS-CoV (2002-2003 outbreak strain) (Kapadia, et al., Virology 2005; 340(2):174-182), human immunodeficiency virus (HIV) (Rose, et al., Cell 2001; 106(5):539-549), hepatitis C virus (HCV) (Ezelle, et al., J. of Virology 2002; 76(23):12325-12334), Lassa virus (LASV) (Cross, et al., J Clin Invest 2020; 130(1):539-551), zika virus (ZIKV) (Emanuel, et al., Sci Rep 2018; 8(1):11043), Crimean-Congo hemorrhagic fever virus (CCHFV) (Rodrigues, et al., Sci Rep 2019; 9(1):7755), to name a few. These VSV-based vaccines generated high antibody titers and protective immunity in murine studies. Most recently, recombinant (rVSV) engineered with the glycoprotein of Zaire Ebola virus (ZEBOV) demonstrated significant protection and was proven safe in clinical trials and ring vaccination. Taken together, these studies suggest that VSV is a robust vaccine platform for generating a respiratory syndrome coronavirus 2 (SARS-CoV-2) vaccine, and in turn, that the VSV vaccine should also demonstrate encouraging safety and immunogenicity data for SARS-CoV-2.

### SUMMARY OF THE INVENTION

Disclosed herein are immunogenic compositions. The immunogenic compositions comprise a vesicular stomatitis virus (VSV). The VSV may express a spike glycoprotein of severe acute respiratory syndrome coronavirus 2 (SARS-CoV-2).

Also disclosed herein are methods for preventing or treating an infection caused by a pathogen. The methods comprise administering to a subject an effective amount of an immunogenic composition comprising a vesicular stomatitis virus. The VSV may express a spike glycoprotein of severe acute respiratory syndrome coronavirus 2 (SARS-CoV-2).

In some embodiments, the VSV serotype is Indiana vesiculovirus or New Jersey vesiculovirus. In some embodiments, the VSV is modified to replace wildtype VSV glycoprotein with the spike glycoprotein of SARS-CoV-2 (COVID19-S).

In some embodiments, the VSV is modified to incorporate a measles hemagglutinin gene, and in some aspects, the measles strain is Edmonston. In some embodiments, the hemagglutinin gene is inserted between the COV-S gene and the VSV-L gene.

In some embodiments, the VSV is modified to incorporate the SARS-CoV-2 nucleocapsid phosphoprotein (COV-N). The COV-N may be incorporated between the COV-S gene and the VSV-L gene.

In some embodiments, the VSV is further modified to incorporate a genetic kill switch. The genetic kill switch may be inserted between the COVID19-S gene and the VSV-L gene.

In some embodiments, the VSV is further modified to incorporate a reporter gene, such as one or more of, a fluorescent gene, a bioluminescent gene, or a gene related to clinical imaging modalities (e.g., sodium iodide symporter (NIS)).

In some embodiments, the immunogenic composition is administered to the subject intramuscularly or via inhalation.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a diagram of the genetic make-up of wild-type (WT) Vesicular Stomatitis Virus (VSV).

FIG. 2 shows a diagram of the genetic make-up of Ebola Zaire vaccine (Ervebo).

FIG. 3 shows a diagram of the genetic make-up of a COVID-19 vaccine described herein (HGI-072).

FIG. 4 shows a diagram of the genetic make-up of a COVID-19 vaccine described herein (HGI-073).

FIG. 5 shows a diagram of the genetic make-up of a COVID-19 vaccine described herein (HGI-074).

FIG. 6 shows infection of HGI-072 at 0 hours, 24 hours, 48 hours, and 72 hours. BHK-21 cells were transfected with human ACE2 cDNA (Sino Biological) using lipofectamine 3000 (ThermoFisher Scientific). Primary antibody: Mouse monoclonal IgG1 SARS-CoV-2 (COVID-19) spike antibody [1A9]; Secondary antibody: IncuCyte Mouse IgG1 FabFluor-488 Antibody; Other: Opti-Green background suppressor.

FIG. 7 provides a graph demonstrating high green intensity & high red intensity on the y-axis and time (hours) on the x-axis. The graph is indicative of colocalization of HGI-072 (green) and human ACE2 receptor (red). Primary antibody: Mouse monoclonal IgG1 SARS-CoV-2 (COVID-19) spike antibody [1A9]; Secondary antibody: IncuCyte Mouse IgG1 FabFluor-488 Antibody; Other: Opti-Green background suppressor.

FIG. 8 shows HGI-072 at Day 3 in VeroC1008 cells in 100% serum free media. 10× magnification, phase and GFP image. Primary antibody: Mouse monoclonal IgG1 SARS-CoV-2 (COVID-19) spike antibody [1A9]; Secondary antibody: IncuCyte Mouse IgG1 FabFluor-488 Antibody; Other: Opti-Green background suppressor.

FIGS. 9A-9C demonstrate virus production in Vero C1008 cells. X axis is time (hours). Y axis is green object count normalized to 00 HRS. FIG. 9A shows virus production in Vero C1008 in 50% serum free media. FIG. 9B shows virus production in Vero C1008 in 75% serum free media. FIG. 9C shows virus production in Vero C1008 in 100% serum free media. Primary antibody: Mouse monoclonal IgG1 SARS-CoV-2 (COVID-19) spike antibody [1A9]; Secondary antibody: IncuCyte Mouse IgG1 FabFluor-488 Antibody; Other: Opti-Green background suppressor.

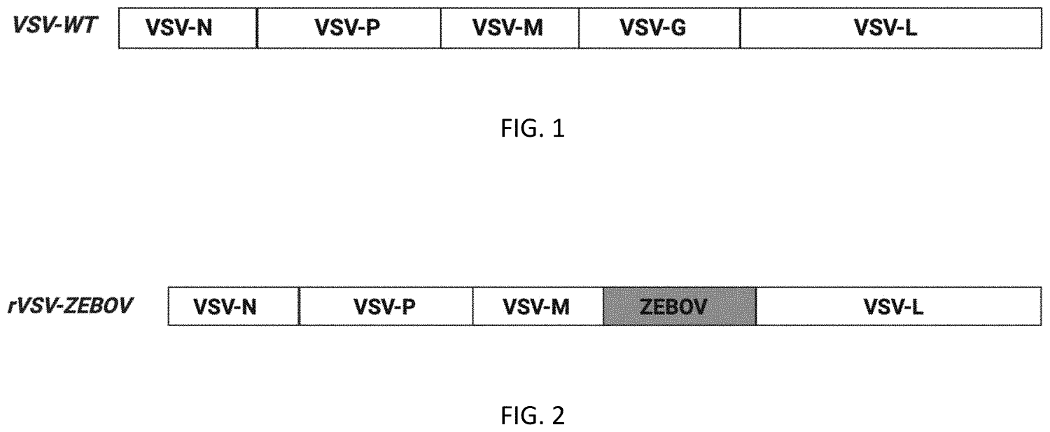

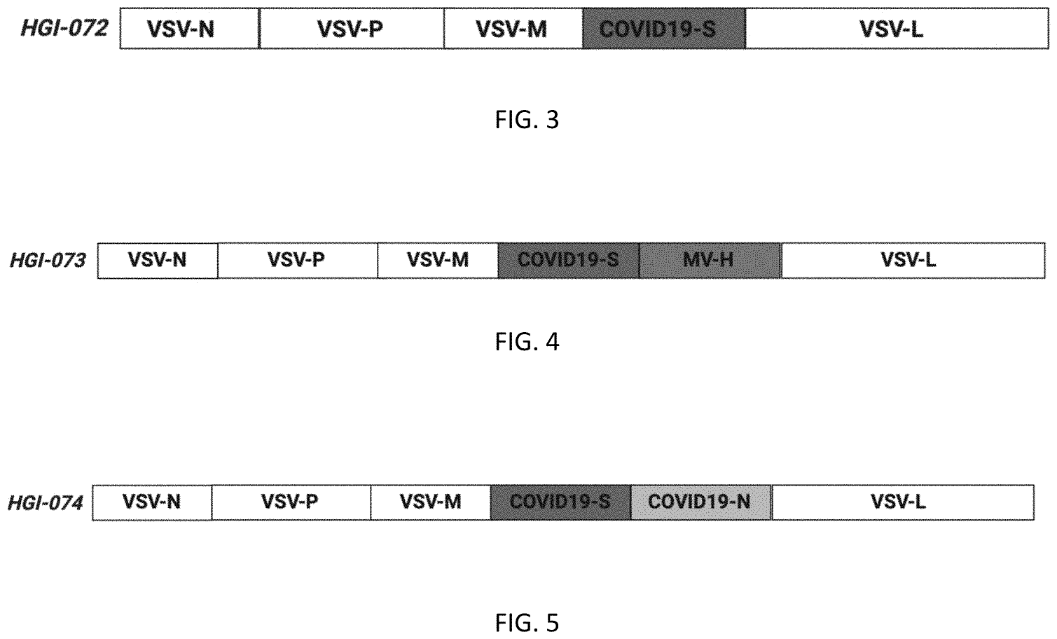

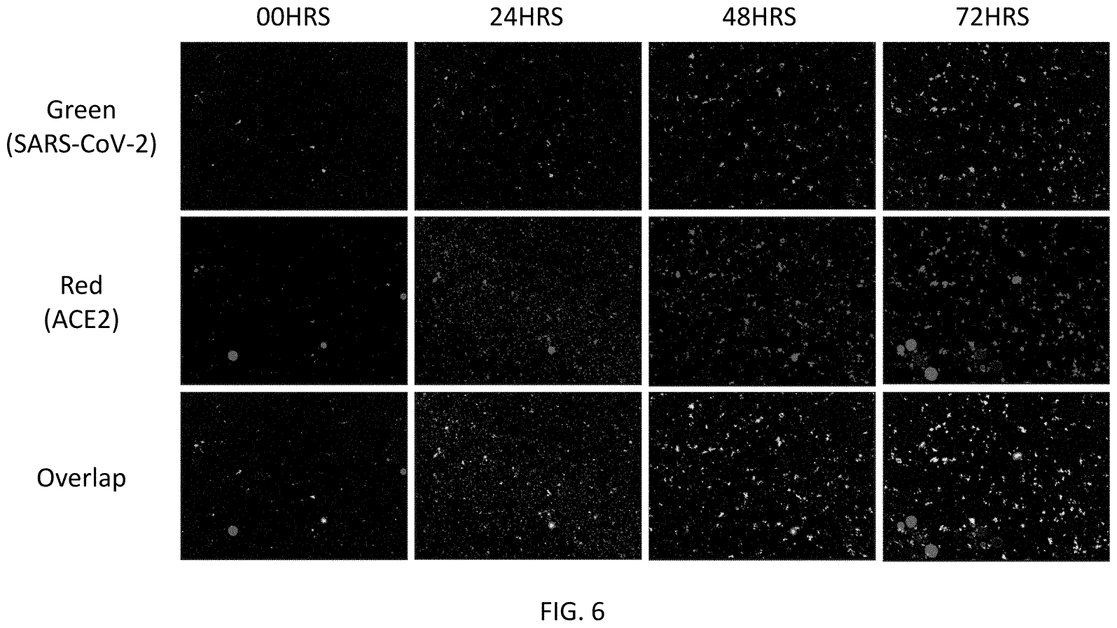

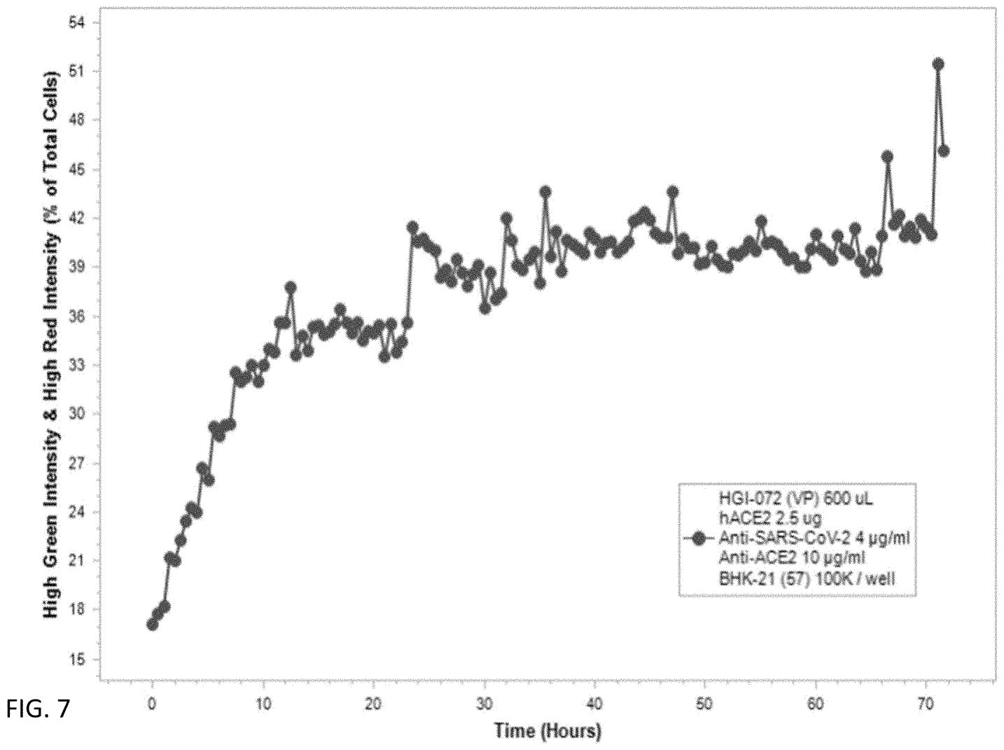

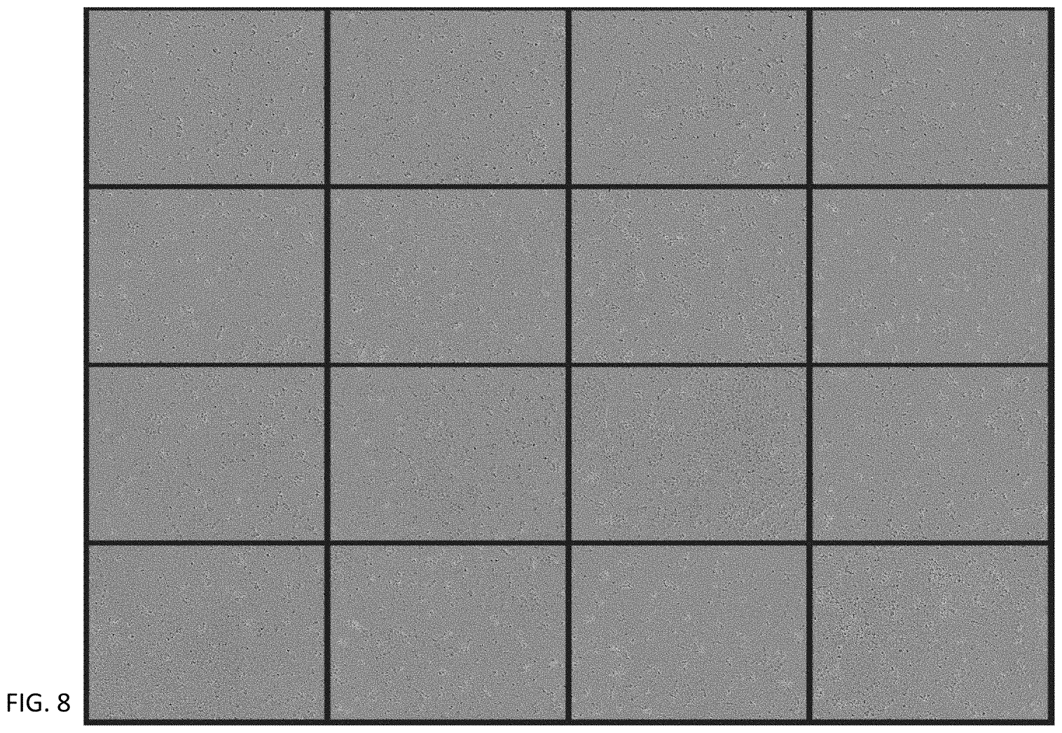

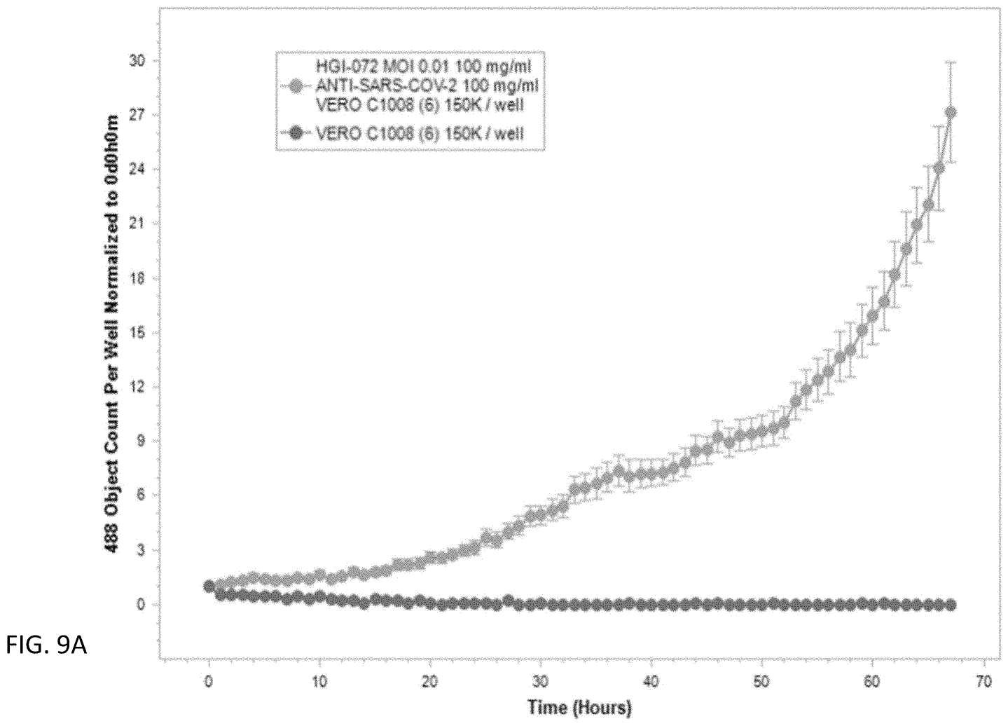

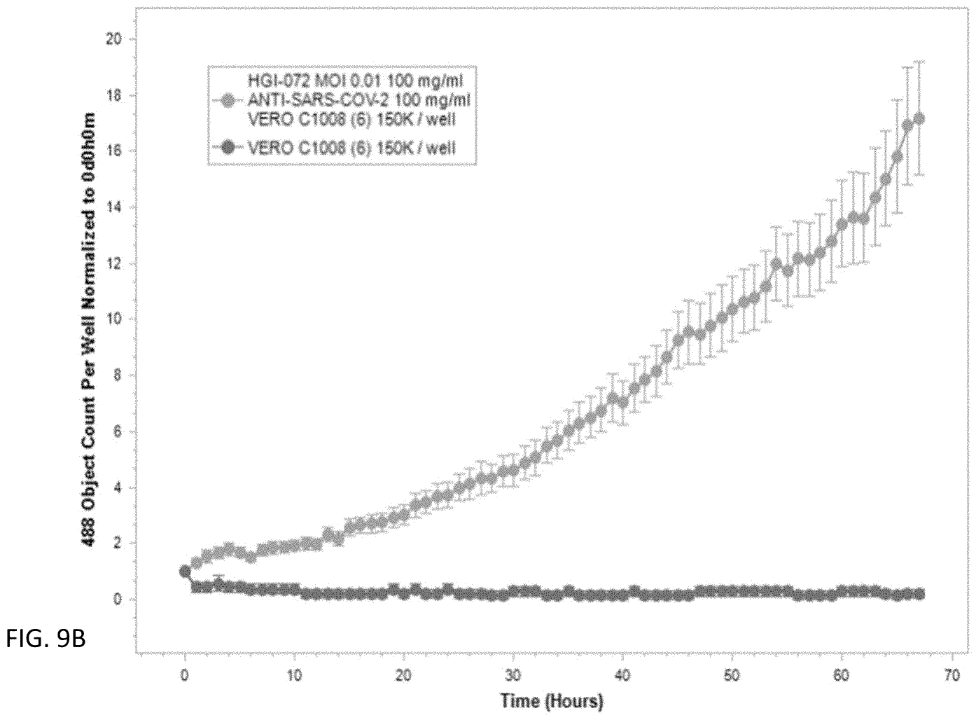

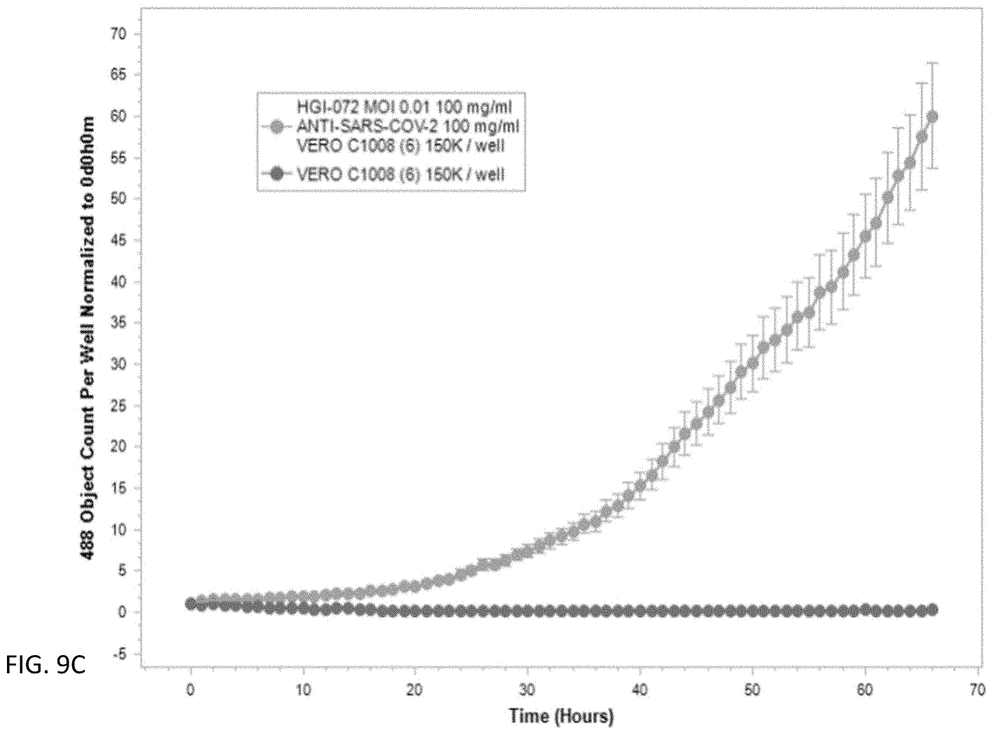

### DETAILED DESCRIPTION OF THE INVENTION

Coronavirus disease 2019 (COVID-19) is an infectious disease caused by severe acute respiratory syndrome coronavirus 2 (SARS-CoV-2). It was first identified in December 2019 in Wuhan, China, and has since spread globally, resulting in an ongoing pandemic. As of May 15 2020, more than 4.47 million cases have been reported across 188 countries and territories, resulting in more than 303,000 deaths. There is an ongoing need for treatments and vaccines to address the ongoing pandemic.

Unless defined otherwise, all technical and scientific terms used herein have the meaning commonly understood by a person skilled in the art. The following references provide one of skill with a general definition of many of the terms used herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

Standard techniques may be used for recombinant DNA, oligonucleotide synthesis, tissue culture and transformation, protein purification, etc. Enzymatic reactions and purification techniques may be performed according to the manufacturer's specifications or as commonly accomplished in the art or as described herein. The following procedures and techniques may be generally performed according to conventional methods well known in the art and as described in various general and more specific references that are cited and discussed throughout the specification. See, e.g., Sambrook et al., 2001, Molecular Cloning: A Laboratory Manuel, 3.sup.rd ed., Cold Spring Harbor Laboratory Press, Cold Spring Harbor, N.Y., which is incorporated herein by reference for any purpose. Unless specific definitions are provided, the nomenclature used in connection with, and the laboratory procedures and techniques of, analytic chemistry, organic chemistry, and medicinal and pharmaceutical chemistry described herein are those well-known and commonly used in the art. Standard techniques may be used for chemical synthesis, chemical analyses, pharmaceutical preparation, formulation, and delivery and treatment of patients.

Immunogenic Compositions

Disclosed herein are immunogenic compositions, e.g., for preventing or treating an infection caused by a pathogen. The immunogenic compositions may comprise a virus. In some embodiments, the virus forms the viral backbone for forming the immunogenic composition. There are numerous viruses that are well known to those skilled in the art and include, but are not limited to, measles virus, rabies virus, Gibbon Ape Leukemia Virus, Sendai Virus, Seneca valley virus, adenovirus (Ad), adeno-associated viruses (AAV), herpes simplex virus (HSV), vaccinia virus (VV), vesicular stomatitis virus (VSV); autonomous parvovirus, myxoma virus (MYXV), Newcastle disease virus (NDV), reovirus, retrovirus, alphaviruses, herpesviruses, influenza virus, Sindbis virus (SINV) or poxvirus, as examples. For example, in some embodiments the virus can be a member of the Rhabdoviridae family, such as from the genus Vesiculovirus. In certain instances, the virus can be Indiana vesiculovirus (VSIV) or New Jersey vesiculovirus (VSNJV). Such viruses, when used as expression vectors are innately non-pathogenic in the selected subjects such as humans or have been modified to render them non-pathogenic in the selected subjects.

As used herein “wild-type” refers to the naturally occurring sequence of a nucleic acid at a genetic locus in the genome of an organism, and sequences transcribed or translated from such a nucleic acid. Thus, the term “wild-type” also may refer to the amino acid sequence encoded by the nucleic acid. As a genetic locus may have more than one sequence or alleles in a population of individuals, the term “wild-type” encompasses all such naturally occurring alleles. As used herein the term “polymorphic” means that variation exists (i.e., two or more alleles exist) at a genetic locus in the individuals of a population. As used herein, “mutant” refers to a change in the sequence of a nucleic acid or its encoded protein, polypeptide, or peptide that is the result of recombinant DNA technology.

A nucleic acid may be made by any technique known to one of ordinary skill in the art. Non-limiting examples of a synthetic nucleic acid, particularly a synthetic oligonucleotide, include a nucleic acid made by in vitro chemical synthesis using phosphotriester, phosphite or phosphoramidite chemistry and solid phase techniques such as described in EP 266,032, or via deoxynucleoside H-phosphonate intermediates as described by Froehler et al., 1986, and U.S. Pat. No. 5,705,629. A non-limiting example of enzymatically produced nucleic acid includes one produced by enzymes in amplification reactions such as PCR™ (see for example, U.S. Pat. Nos. 4,683,202 and 4,682,195), or the synthesis of oligonucleotides described in U.S. Pat. No. 5,645,897. A non-limiting example of a biologically produced nucleic acid includes recombinant nucleic acid production in living cells, such as recombinant DNA vector production in bacteria (see for example, Sambrook et al. 1989).

The nucleic acid(s), regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). The overall length may vary considerably between nucleic acid constructs. Thus, a nucleic acid segment of almost any length may be employed, with the total length preferably being limited by the ease of preparation or use in the intended recombinant nucleic acid protocol.

As used herein the terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid can be single-stranded, or partially or completely double-stranded (duplex). Duplex nucleic acids can be homoduplex or heteroduplex.

The terms “protein,” “peptide,” “polypeptide,” and “amino acid sequence” are used interchangeably herein to refer to polymers of amino acid residues of any length. The polymer may be linear or branched, it may comprise modified amino acids or amino acid analogs, and it may be interrupted by chemical moieties other than amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling or bioactive component.

As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

As used herein the term “transgene” may be used to refer to “recombinant” nucleotide sequences that may be derived from any of the nucleotide sequences encoding the proteins of the present invention. The term “recombinant” means a nucleotide sequence that has been manipulated “by man” and which does not occur in nature, or is linked to another nucleotide sequence or found in a different arrangement in nature. It is understood that manipulated “by man” means manipulated by some artificial means, including by use of machines, codon optimization, restriction enzymes, etc.

By “expression construct” or “expression cassette” is meant a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide to be delivered to a host cell, either in vitro or in vivo. A “plasmid,” a common type of a vector, is an extra-chromosomal DNA molecule separate from the chromosomal DNA that is capable of replicating independently of the chromosomal DNA. In certain cases, it is circular and double-stranded.

Any vector that allows expression of the antigens described herein may be used in accordance with the present invention. In certain embodiments, the antigens may be used in vitro (such as using cell-free expression systems) and/or in cultured cells grown in vitro in order to produce the encoded SARS-CoV-2-antigens which may then be used for various applications such as in the production of proteinaceous vaccines. For such applications, any vector that allows expression of the antigens in vitro and/or in cultured cells may be used.

In other embodiments, where it is desired that the antigens be expressed in vivo, for example when the transgenes of the invention are used in DNA or DNA-containing vaccines, any vector that allows for the expression of the antigens described herein, and is safe for use in vivo, may be used. In preferred embodiments the vectors used are safe for use in humans, mammals and/or laboratory animals.

The term “promoter” is used herein in its ordinary sense to refer to a nucleotide region comprising a DNA regulatory sequence, wherein the regulatory sequence is derived from a gene that is capable of binding RNA polymerase and initiating transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

By “operably linked” or co-expressed” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” or “co-expressed” with reference to peptide and/or polypeptide molecules means that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of heterologous molecules.

Described herein are immunogenic compositions comprising recombinant vesicular stomatitis viruses (VSV) and recombinant VSV particles expressing foreign glycoproteins, for example, viral glycoproteins. In alternative embodiments, an immunogenic composition comprises a recombinant measles virus and recombinant measles particles expressing foreign glycoproteins, for example, viral glycoproteins. Also described is the use of the recombinant particles to induce an immune response in an animal in need thereof.

In some aspects, a recombinant vesicular stomatitis virus (VSV) vector is used to produce an immunogenic composition. VSV is a practical, safe, and immunogenic vector for conducting animal studies, and an attractive candidate for developing vaccines for use in humans. VSV is a member of the Rhabdoviridae family of enveloped viruses containing a nonsegmented, negative-sense RNA genome. The genome is composed of 5 genes arranged sequentially 3′-N-P-M-G-L-S′, each encoding a polypeptide found in mature virions. Notably, the surface glycoprotein G is a transmembrane polypeptide that is present in the viral envelope as a homotrimer and it mediates cell attachment and infection.

In some embodiments, recombinant VSV expresses the native VSV glycoprotein and additional genes coding for foreign glycoprotein genes are added. Such viruses have the host range of VSV, but may express the foreign glycoprotein genes during replication. In some embodiments, such viruses may be used as glycoprotein replacement viruses. In alternative embodiments, wild-type VSV is modified to replace the VSV-glycoprotein with a foreign glycoprotein.

In certain embodiments, the foreign glycoprotein is a spike glycoprotein from a coronavirus, such as severe acute respiratory syndrome coronavirus 2 (SARS-CoV-2). In other embodiments, immunogenic fragments of these glycoproteins may be used, as may fusion proteins including immunogenic fragments or epitopes of the glycoprotein of interest. As will be appreciated by one of skill in the art, there are numerous algorithms and/or computer programs available for predicting potentially immunogenic fragments and epitopes of the glycoprotein of interest.

In one embodiment, using VSV as the viral backbone, the VSV-glycoprotein (VSV-G) is replaced with SARS-CoV-2 spike glycoprotein (COVID19-S or COV-S). In alternative embodiments, measles may be used as the viral backbone and may be modified to express the SARS-CoV-2 spike glycoprotein.

In some embodiments, the gene order in the full length VSV genome clone may be altered such that the first gene will code for the glycoprotein rather than the nucleoprotein. This may have two effects: the virus may be further attenuated, and more glycoprotein may be made, thereby increasing the efficacy of the vaccine.

As will be apparent to one of skill in the art, only the foreign glycoprotein will be expressed on the surface of the recombinant VSV particle (generally referred to as pseudotyping) and is thus presented to the host immune system. Thus, the recombinant VSV particle is an infectious system that simulates infection with the foreign virus and yet does not cause disease or the symptoms associated with the foreign virus. Furthermore, the immune response generated is protective regardless of the route of immunization. As will be apparent to one of skill in the art, only a single dose of the vaccine is required to elicit a protective immune response in the host, which may be a human.

In some embodiments, a second foreign viral protein is inserted in addition to the glycoprotein gene, thereby making a multivalent recombinant viral particle. In some embodiments, a VSV modified to express COV-S is further modified to incorporate a nucleocapsid phosphoprotein. For example, the nucleocapsid protein may be a SARS-CoV-2 nucleocapsid phosphoprotein (COV-N). In one embodiment, COV-N is inserted between the COV-S gene and the VSV-L gene. In some embodiments, a VSV modified to express COV-S is further modified to incorporate a measles hemagglutinin gene (MV-H). The hemagglutinin gene may be from the measles virus strain Edmonston. In one embodiment, the hemagglutinin gene is inserted between the COV-S gene and the VSV-L gene.

In some embodiments VSV is further modified to include a kill switch, e.g., a genetic kill switch, and/or a reporter gene. A reporter gene may be any reporter gene known to those of skill in the art, including, but not limited to, bioluminescent genes (e.g., Luc), fluorescent genes (e.g., RFP, GFP, BFP, DsRed, mCherry, EGFP, EBFP, TxRed, moxGFP, moxBFP, tdTomato), and genes related to clinical imaging modalities (e.g., sodium iodide symporter (NIS)). A kill switch and/or a reporter gene may be added to any of the recombinant VSV vectors described herein. In one embodiment, a kill switch is inserted between the COV-S gene and the VSV-L gene. In one embodiment, a reporter gene is inserted between the COV-S gene and the VSV-L gene. In certain embodiments, where additional genes (e.g., COV-N or MV-H) have been added to VSV, a kill switch or reporter gene will be inserted before VSV-L. For example, a VSV may be modified to include COV-S/MV-H/Kill Switch/VSV-L or COV-S/COV-N/Kill Switch/VSV-L. In other examples, a VSV is modified to include COV-S/MV-H/Reporter/VSV-L or COV-S/COV-N/Reporter/VSV-L.

Methods of Treatment

Some embodiments of the present invention relate to methods of prevention and/or treatment of an infection caused by a pathogen, such as coronavirus disease 2019 (COVID 19), by the delivery of one or more immunogenic compositions described herein. An effective amount of the immunogenic composition is an amount sufficient to prevent an infection in a subject to whom the composition is administered, or an amount that limits the severity of an infection in a subject to whom the composition is administered, and/or or to treat an infection in a subject to whom the composition is administered.

As used herein, the terms “treat” and “treating” refers to a treatment/therapy from which a subject receives a beneficial effect, such as the reduction, decrease, attenuation, diminishment, stabilization, suppression, inhibition or arrest of the development or progression of a condition or disease (e.g., an infection), or a symptom thereof “Treating” a condition or disease refers to curing as well as ameliorating at least one symptom of the condition or disease, and includes administration of a composition which reduces the frequency of, or delays the onset of, symptoms of a medical condition in a subject in need relative to a subject which does not receive the composition. “Treatment” as used herein covers any treatment of a disease or condition of a mammal, particularly a human, and includes: (a) preventing symptoms of the disease or condition from occurring in a subject which may be predisposed to the disease or condition but has not yet begun experiencing symptoms; (b) inhibiting the disease or condition (e.g., arresting its development); or (c) relieving the disease or condition (e.g., causing regression of the disease or condition, providing improvement in one or more symptoms).

The term “treatment” may include prophylaxis, and may further include the implementation of measures intended to reduce the subject's likelihood of contracting an infection (e.g., COVID-19). For example, in certain aspects, the treatment may include medical and/or pharmacologic interventions intended to reduce the subject's likelihood of contracting an infection (e.g., the prophylactic administration of one or more vaccines, convalescent plasma infusion and/or antibodies to the subject). In other embodiments, the treatment may include non-pharmacologic interventions, such as self-isolation and/or quarantine of a subject identified as being susceptible to developing an infection and/or the associated complications. Alternatively, treatment is “effective” if the progression of a disease is reduced or halted.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. Preferably, the patient is a human (Homo sapiens). The subject may be of any gender.

When provided prophylactically, the immunogenic compositions of the invention are ideally administered to a subject in advance of infection (e.g., SARS-CoV-2 infection), or evidence of infection, or in advance of any symptom due to coronavirus (e.g., COVID-19), especially in high-risk subjects. The prophylactic administration of the immunogenic compositions can serve to provide protective immunity of a subject against SARS-CoV-2 infection or to prevent or attenuate the progression of COVID-19 in a subject already infected with SARS-CoV-2. When provided therapeutically, the immunogenic compositions can serve to ameliorate and treat COVID-19 symptoms and are advantageously used as soon after infection as possible, preferably before appearance of any symptoms of COVID-19 but may also be used at (or after) the onset of the disease symptoms.

The immunogenic compositions can be administered using any suitable delivery method including, but not limited to, orally, intramuscular, intravenous, intradermal, subcutaneously, intraperitoneally, intranasally, mucosal, and topical delivery. Such techniques are well known to those of skill in the art. Other examples include, delivery of DNA to animal tissue by cationic liposomes (Watanabe et al., (1994) Mol. Reprod. Dev. 38:268-274; and WO 96/20013), direct injection of naked DNA into animal muscle tissue (Robinson et al., (1993) Vaccine 11:957-960; Hoffman et al., (1994) Vaccine 12: 1529-1533; Xiang et al., (1994) Virology 199: 132-140; Webster et al., (1994) Vaccine 12: 1495-1498; Davis et al., (1994) Vaccine 12: 1503-1509; and Davis et al., (1993) Hum. Mol. Gen. 2: 1847-1851), or intradermal injection of DNA using “gene gun” technology (Johnston et al., (1994) Meth. Cell Biol. 43:353-365).

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The phrase “pharmaceutically-acceptable” or “pharmacologically-acceptable” refers to molecular entities and compositions that do not produce an allergic or similar untoward reaction when administered to a subject. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Except insofar as any conventional media or agent is incompatible with the viral agent, its use in the therapeutic compositions is contemplated. Supplementary active ingredients can also be incorporated into the compositions.

The treatments may include various “unit doses” defined as containing a predetermined-quantity of the therapeutic composition. The quantity to be administered, and the particular route and formulation, are within the skill of those in the clinical arts. Unit dose of the present invention may conveniently be described in terms of plaque forming units (pfu) for a viral construct.

In some embodiments, the immunogenic compositions of the invention are administered in vivo, for example where the aim is to produce an immunogenic response in a subject. A “subject” in the context of the present invention may be any animal. For example, in some embodiments it may be desired to express the transgenes of the invention in a laboratory animal, such as for pre-clinical testing of the SARS-CoV-2 immunogenic compositions and vaccines of the invention. In other embodiments, it will be desirable to express the antigens of the invention in human subjects, such as in clinical trials and for actual clinical use of the immunogenic compositions and vaccine of the invention. In preferred embodiments the subject is a human, for example a human that is infected with, or is at risk of infection with, SARS-CoV-2.

For such in vivo applications the immunogenic composition is administered in admixture with a pharmaceutically acceptable carrier. The immunogenic compositions of the invention are useful to stimulate an immune response against SARS-CoV-2 and may be used as one or more components of a prophylactic or therapeutic vaccine against SARS-CoV-2 for the prevention, amelioration or treatment of COVID-19. The nucleic acids and vectors of the invention are particularly useful for providing genetic vaccines, i.e. vaccines for delivering the nucleic acids encoding the antigens of the invention to a subject, such as a human, such that the antigens are then expressed in the subject to elicit an immune response.

In some embodiments, the immunogenic compositions are formulated as injectable suspensions, solutions, sprays, lyophilized powders, syrups, elixirs and the like. Any suitable form of immunogenic composition may be used. To prepare such a composition, a nucleic acid or vector of the invention, having the desired degree of purity, is mixed with one or more pharmaceutically acceptable carriers and/or excipients. The carriers and excipients must be “acceptable” in the sense of being compatible with the other ingredients of the composition. Acceptable carriers, excipients, or stabilizers are nontoxic to recipients at the dosages and concentrations employed, and include, but are not limited to, water, saline, phosphate buffered saline, dextrose, glycerol, ethanol, or combinations thereof, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, an immunogenic composition is formulated in the form of an oil-in-water emulsion. The oil-in-water emulsion can be based, for example, on light liquid paraffin oil (European Pharmacopea type); isoprenoid oil such as squalane, squalene, EICOSANE™ or tetratetracontane; oil resulting from the oligomerization of alkene(s), e.g., isobutene or decene; esters of acids or of alcohols containing a linear alkyl group, such as plant oils, ethyl oleate, propylene glycol di(caprylate/caprate), glyceryl tri(caprylate/caprate) or propylene glycol dioleate; esters of branched fatty acids or alcohols, e.g., isostearic acid esters. The oil advantageously is used in combination with emulsifiers to form the emulsion. The emulsifiers can be nonionic surfactants, such as esters of sorbitan, mannide (e.g., anhydromannitol oleate), glycerol, polyglycerol, propylene glycol, and oleic, isostearic, ricinoleic, or hydroxystearic acid, which are optionally ethoxylated, and polyoxypropylene-polyoxyethylene copolymer blocks, such as the Pluronic® products, e.g., L121. The adjuvant can be a mixture of emulsifier(s), micelle-forming agent, and oil such as that which is commercially available under the name Provax® (IDEC Pharmaceuticals, San Diego, Calif.).

The immunogenic compositions described herein can contain additional substances, such as wetting or emulsifying agents, buffering agents, or adjuvants to enhance the effectiveness of the vaccines (Remington's Pharmaceutical Sciences, 18th edition, Mack Publishing Company, (ed.) 1980).

In some embodiments, the immunogenic compositions are designed to introduce the nucleic acids or expression vectors to a desired site of action and release it at an appropriate and controllable rate. Methods of preparing controlled-release formulations are known in the art. For example, controlled release preparations can be produced by the use of polymers to complex or absorb the immunogen and/or immunogenic composition. A controlled-release formulations can be prepared using appropriate macromolecules (for example, polyesters, polyamino acids, polyvinyl, pyrrolidone, ethylenevinylacetate, methylcellulose, carboxymethylcellulose, or protamine sulfate) known to provide the desired controlled release characteristics or release profile. Another possible method to control the duration of action by a controlled-release preparation is to incorporate the active ingredients into particles of a polymeric material such as, for example, polyesters, polyamino acids, hydrogels, polylactic acid, polyglycolic acid, copolymers of these acids, or ethylene vinylacetate copolymers. Alternatively, instead of incorporating these active ingredients into polymeric particles, it is possible to entrap these materials into microcapsules prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nano-particles and nanocapsules) or in macroemulsions. Such techniques are disclosed in New Trends and Developments in Vaccines, Voller et al. (eds.), University Park Press, Baltimore, Md., 1978 and Remington's Pharmaceutical Sciences, 16th edition.

Suitable dosages of the nucleic acids and expression vectors of the invention (collectively, the immunogens) in the immunogenic composition of the invention can be readily determined by those of skill in the art. For example, the dosage of the immunogens can vary depending on the route of administration and the size of the subject. Suitable doses can be determined by those of skill in the art, for example by measuring the immune response of a subject, such as a laboratory animal, using conventional immunological techniques, and adjusting the dosages as appropriate. Such techniques for measuring the immune response of the subject include but are not limited to, chromium release assays, tetramer binding assays, IFN-γ ELISPOT assays, IL-2 ELISPOT assays, intracellular cytokine assays, and other immunological detection assays, e.g., as detailed in the text “Antibodies: A Laboratory Manual” by Ed Harlow and David Lane.

Immunization schedules (or regimens) are well known for animals (including humans) and can be readily determined for the particular subject and immunogenic composition. Hence, the immunogens can be administered one or more times to the subject. Preferably, there is a set time interval between separate administrations of the immunogenic composition. While this interval varies for every subject, typically it ranges from 7 days to several weeks, and is often 2, 4, 6 or 8 weeks. For humans, the interval is typically from 2 to 6 weeks. The immunization regimes typically have from 1 to 6 administrations of the immunogenic composition, but may have as few as one or two or four. The methods of inducing an immune response can also include administration of an adjuvant with the immunogens. In some instances, annual, biannual or other long interval (5-10 years) booster immunization can supplement the initial immunization protocol.

Combination Therapies

The compositions and methods of the present invention may be used in the context of infections including viral infections, such as COVID-19. In order to increase the effectiveness of a treatment with the compositions of the present invention, it may be desirable to combine these compositions with other agents effective in the treatment of those diseases and conditions. For example, the treatment of an infection may be implemented with immunogenic compositions of the present invention in combination with other SARS-CoV-2 immunogens and/or SARS-CoV-2 immunogenic compositions, e.g., with “other” immunological, antigenic or vaccine or therapeutic compositions thereby providing multivalent or “cocktail” or combination compositions of the invention and methods of employing them.

As used herein, the term “in combination” in the context of the administration of (a) therapy(ies) to a subject, refers to the use of more than one therapy. The use of the term “in combination” does not restrict the order in which therapies are administered to a subject. A first therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

Administration of the immunogenic compositions of the present invention to a patient in combination with a secondary therapy will follow general protocols for the administration of that particular secondary therapy, repeating treatment cycles as necessary. The ingredients and manner (sequential or co-administration) of administration, as well as dosages, can be determined taking into consideration such factors as the age, sex, weight, species and condition of the particular subject, and the route of administration.

When used in combination, the other SARS-CoV-2 immunogens can be administered at the same time or at different times as part of an overall immunization regime, e.g., as part of a prime-boost regimen or other immunization protocol.

The inventions disclosed herein will be exemplified in a non-limiting manner by the following. The teachings of all references, patents and patent applications cited herein are incorporated fully by reference herein.

### EXEMPLIFICATION

Cell Culture

BHK-21 cell line was purchased from ATCC (CCL-10). The cells were maintained in DMEM (Corning) supplemented with 10% FBS (Gibco), 5% Penicillin/Streptomycin (Corning), and 1% Amphotericin B (Corning) at 37° C. and 5% CO2.

Genome Assembly

The complete genome design was deconstructed into 0.3-1.8 kb fragments, which were manufactured via Twist Bioscience's silicon-based technology platform. Following DNA synthesis, 1000 ng sequence verified gene fragments were resuspended in 100 uL of nuclease-free water (NFW) and amplified via Multiple Displacement Amplification (MDA) with the REPLI-g Whole Genome Amplification kit (Qiagen).

To construct full-length virus genomes, amplified fragments were assembled on ice via homologous recombination. The reaction contained 0.15 pmol of mini fragments (<3000 bp), 0.05 pmol of mega fragments (>3000 bp), and commercial DNA assembly master mix at a concentration ranging from 1:10 to 1:2 of the total reaction volume. The reaction tube/plate was vortexed briefly, centrifuged for 10 seconds at 2000 G, and run on a thermocycler for 30 minutes at 65 C. Samples were cooled to 4 C and then used in transfection or stored at −20 C.

Transfection

BHK-21 cells (ATCC) were plated in cell culture flasks or plates with 6, 12, 24, 48, or 96 wells (Corning) with complete DMEM media. After 24 hours, the cells were washed with PBS (lx) and (co)transfected with human angiotensin I converting enzyme 2 (hACE2) ORF mammalian expression plasmid with a C-His tag (Sino Biological) and/or the complete genome of artificial VSV-based vaccine constructs (HGI-072, HGI-073, HGI-074) using Lipofectamine 3000 (Invitrogen). During this process, the cells were cultured with DMEM supplemented with 5% FBS. By 72 hours post-transfection, virus particles were collected, freeze-thawed with liquid nitrogen (3×), and stored in −80 C until needed.

Immunocytochemistry

Immunocytochemistry (ICC) and immunofluorescence (IF) staining were performed to confirm transfection efficiency and virus production. Cells were seeded at an appropriate density (20-30% confluence) and incubated overnight. SARS-CoV-2 (COVID-19) spike antibody [1A9] (GeneTex) was diluted to 1:2000 and mixed with IncuCyte FabFluor-488 Antibody Labeling reagent (Essen Bioscience) and cell growth media in a separate tube or cell culture plate at a 1:3 molar ratio (antibody:FabFluor-488) for 15 minutes. Then, samples were incubated with labeled antibody to confirm viral bootup and the production of functional virus particles. IncuCyte Opti-Green background suppressor was added to cell media for a final dilution of 1:200. To confirm hACE2 expression, cells were stained with rabbit anti-ACE2 antibody (Abcam) at a concentration of 10 μg/mL and Alexa Fluor 594 goat anti-rabbit IgG (Invitrogen) at a dilution of 2 μg/mL.

IncuCyte Live-Cell Analysis system (Sartorius) was used for real-time visualization and analysis. Red fluorescence, green fluorescence, and HD phase contrast images (3-9 per well) were acquired every 30 minutes to 1 hour at 10×-20× magnification throughout the duration of the experiment. Cell count and confluence percentage were assessed to monitor cell health and viability. Green object counts (number per image or well), total area (μm²/image), and intensity (RCU×μm²/image) were measured to determine virus growth and kinetics. Measurements for red object count, total area, and intensity were taken to evaluate hACE2 expression. Overlap of virus and hACE2 was calculated to assess colocalization and receptor binding. Mean and standard deviation were calculated for each sample/well and graphed over time (hours).

Viral Vaccine Manufacturing

Vero-SF-ACF cells were purchased from ATCC (CCL-81.5) and maintained in NutriVero Flex10 media (Biological Industries) supplemented with 4 mM L-Glutamine (ATCC) at 37° C. and 5% CO2. Derived from the parental Vero cell line (ATCC CCL-81), this cell line is adapted to serum-free (SF) and animal-component free (ACF) media.

Cells were transfected with hACE2 ORF mammalian expression plasmid with a C-His tag (Sino Biological). Hygromycin B was used for the selection of hACE2 expressing vero cells (ACE2/Vero). Following selection, ACE2/vero cells were expanded.

The virus will be expanded using methods generally known to those of skill in the art. Serum free adapted vero cells (these are adherent) will be infected with HGI-072 (virus) at 24 hours post-plating, and viruses will be allowed to replicate for 24-96 hours post-infection. The expansion phase will be conducted at 34 C-37 C (temperature).

Upon completion of the virus expansion, the virus or components thereof are harvested from the cell culture. This can be done by routine methods, which are as such known to the skilled person. The supernatant may be aspirated, centrifuged (anywhere from 250 to 5000 g for anywhere from 2 mins to 30 mins to pellet debris and/or induce cell lysis), and aliquoted. Physical means (e.g. cell scrapers) or chemical means (e.g. trypsin, trypLE) may be used in some aspects to induce cell lifting from the flask and lysis. Benzonase (anywhere from 1 to 100 Units) may be used in some aspects to degrade nucleic acids, further removing debris in cell suspension. A cell strainer may be used to remove debris.

The harvest may be clarified by methods known to those of skill in the art, and then purified. The resulting concentrated virus suspension can optionally be diluted to a desired concentration.

Exemplary VSV

An example of an artificial vesicular stomatitis virus expressing the spike glycoprotein of severe acute respiratory syndrome coronavirus 2, complete genome, is identified as HGI-072.1.

Features:

| | |
|---|---|
| mRNA | 51..1376 |
| | /product = ″N mRNA″ |
| | /note = ″Nucleocapsid″ |
| | /reference = ″J02428.1″ |
| mRNA | 1386..2199 |
| | /product = ″P mRNA″ |
| | /note = ″Phosphoprotein″ |
| | /reference = ″J02428.1″ |
| mRNA | 2209..3039 |
| | /product = ″M mRNA″ |
| | /note = ″Matrix″ |
| | /reference = ″J02428.1″ |
| Variation | 3047..3094 |
| | /product = ″VAl″ |
| | /note = ″Viral Adaptor 1″ |
| | /reference = ″HGI-007.1″ |
| Variation | 3095..3096 |
| | /product = ″GB1″ |
| | /note = ″Gene Boundary 1″ |
| UTR | 3097..3125 |
| | /product = ″VSV-G UTR″ |
| | /note = ″VSV Glycoprotein UTR″ |
| | /reference = ″J02428.1″ |
| mRNA | 3126..3173 |
| | /product = ″Signal Peptide″ |
| | /note = ″VSV-G Signal peptide″ |
| | /reference = ″J02428.1″ |
| CDS | 3174..6995 |
| | /product = ″COVID-S″ |
| | /note = ″SARS-CoV-2 Spike Glycoprotein″ |
| | /reference = ″NC_045512.2″ |
| mRNA | 6996..7004 |
| | /product = ″VSV-G Stop″ |
| | /note = ″VSV-G Consensus Stop Sequence″ |
| | /reference = ″J02428.1″ |
| Variation | 7005..7046 |
| | /product = ″VA2″ |
| | /note = ″Viral Adaptor 2″ |
| | /reference = ″HGI-007.1″ |
| Variation | 7047..7103 |
| | /product = ″VA4″ |
| | /note = ″Viral Adaptor 4″ |
| | /reference = ″HGI-007.1″ |
| mRNA | 7104..13476 |
| | /product = ″L mRNA″ |
| | /note = ″Polymerase″ |
| | /reference = ″J02428.1″ |

ORIGIN:
ACGAAGACAAACAAACCATTATTATCATTAAAAGGCTCAGGAGAAACTTT
AACAGTAATCAAAATGTCTGTTACAGTCAAGAGAATCATTGACAACACAG
TCATAGTTCCAAAACTTCCTGCAAATGAGGATCCAGTGGAATACCCGGCA
GATTACTTCAGAAAATCAAAGGAGATTCCTCTTTACATCAATACTACAAA
AAGTTTGTCAGATCTAAGAGGATATGTCTACCAAGGCCTCAAATCCGGAA
ATGTATCAATCATACATGTCAACAGCTACTTGTATGGAGCATTAAAGGAC
ATCCGGGGTAAGTTGGATAAAGATTGGTCAAGTTTCGGAATAAACATCGG
GAAAGCAGGGGATACAATCGGAATATTTGACCTTGTATCCTTGAAAGCCC
TGGACGGCGTACTTCCAGATGGAGTATCGGATGCTTCCAGAACCAGCGCA
GATGACAAATGGTTGCCTTTGTATCTACTTGGCTTATACAGAGTGGGCAGA
ACACAAATGCCTGAATACAGAAAAAAGCTCATGGATGGGCTGACAAATC
AATGCAAAATGATCAATGAACAGTTTGAACCTCTTGTGCCAGAAGGTCGT
GACATTTTTGATGTGTGGGGAAATGACAGTAATTACACAAAAATTGTCGC
TGCAGTGGACATGTTCTTCCACATGTTCAAAAAACATGAATGTGCCTCGTT
CAGATACGGAACTATTGTTTCCAGATTCAAAGATTGTGCTGCATTGGCAAC
ATTTGGACACCTCTGCAAAATAACCGGAATGTCTACAGAAGATGTAACGA
CCTGGATCTTGAACCGAGAAGTTGCAGATGAAATGGTCCAAATGATGCTT
CCAGGCCAAGAAATTGACAAGGCCGATTCATACATGCCTTATTTGATCGA
CTTTGGATTGTCTTCTAAGTCTCCATATTCTTCCGTCAAAAACCCTGCCTT
CCACTTCTGGGGGCAATTGACAGCTCTTCTGCTCAGATCCACCAGAGCAAG
GAATGCCCGACAGCCTGATGACATTGAGTATACATCTCTTACTACAGCAG
GTTTGTTGTACGCTTATGCAGTAGGATCCTCTGCCGACTTGGCACAACAGT
TTTGTGTTGGAGATAACAAATACACTCCAGATGATAGTACCGGAGGATTG
ACGACTAATGCACCGCCACAAGGCAGAGATGTGGTCGAATGGCTCGGATG
GTTTGAAGATCAAAACAGAAAACCGACTCCTGATATGATGCAGTATGCGA
AAAGAGCAGTCATGTCACTGCAAGGCCTAAGAGAGAAGACAATTGGCAA
GTATGCTAAGTCAGAATTTGACAAATGACCCTATAATTCTCAGATCACCTA
TTATATATTATGCTACATATGAAAAAAACTAACAGATATCATGGATAATCT
CACAAAAGTTCGTGAGTATCTCAAGTCCTATTCTCGTCTGGATCAGGCGGT
AGGAGAGATAGATGAGATCGAAGCACAACGAGCTGAAAAGTCCAATTAT
GAGTTGTTCCAAGAGGATGGAGTGGAAGAGCATACTAAGCCCTCTTATTT
TCAGGCAGCAGATGATTCTGACACAGAATCTGAACCAGAAATTGAAGACA
ATCAAGGTTTGTATGCACAGGATCCAGAAGCTGAGCAAGTTGAAGGCTTT
ATACAGGGGCCTTTAGATGACTATGCAGATGAGGAAGTGGATGTTGTATT
TACTTCGGACTGGAAACCACCTGAGCTTGAATCTGACGAGCATGGAAAGA
CCTTACGGTTGACATCGCCAGAGGGTTTAAGTGGAGAGCAGAAATCCCAG
TGGCTTTCGACGATTAAAGCAGTCGTGCAAAGTGCCAAATACTGGAATCT
GGCAGAGTGCACATTTGAAGCATCGGGAGAAGGGGTCATTATGAAGGAG
CGCCAGATAACTCCGGATGTATATAAGGTCACTCCAGTGATGAACACACA
TCCGTCCCAATCAGAAGCAGTATCAGATGTTTGGTCTCTCTCAAAGACATC
CATGACTTTCCAACCCAAGAAAGCAAGTCTTCAGCCTCTCACCATATCCTT
GGATGAATTGTTCTCATCTAGAGGAGAGTTCATCTCTGTCGGAGGTGACG
GACGAATGTCTCATAAAGAGGCCATCCTGCTCGGCCTGAGATACAAAAAG
TTGTACAATCAGGCGAGAGTCAAATATTCTCTGTAGACTATGAAAAAAAG
TAACAGATATCACGATCTAAGTGTTATCCCAATCCATTCATCATGAGTTCC
TTAAAGAAGATTCTCGGTCTGAAGGGGAAAGGTAAGAAATCTAAGAAATT
AGGGATCGCACCACCCCCTTATGAAGAGGACACTAGCATGGAGTATGCTC
CGAGCGCTCCAATTGACAAATCCTATTTTGGAGTTGACGAGATGGACACC
TATGATCCGAATCAATTAAGATATGAGAAATTCTTCTTTACAGTGAAAATG
ACGGTTAGATCTAATCGTCCGTTCAGAACATACTCAGATGTGGCAGCCGC
TGTATCCCATTGGGATCACATGTACATCGGAATGGCAGGGAAACGTCCCT
TCTACAAAATCTTGGCTTTTTTGGGTTCTTCTAATCTAAAGGCCACTCCAG
CGGTATTGGCAGATCAAGGTCAACCAGAGTATCACACTCACTGCGAAGGC
AGGGCTTATTTGCCACATAGGATGGGGAAGACCCCTCCCATGCTCAATGT
ACCAGAGCACTTCAGAAGACCATTCAATATAGGTCTTTACAAGGGAACGA
TTGAGCTCACAATGACCATCTACGATGATGAGTCACTGGAAGCAGCTCCT
ATGATCTGGGATCATTTCAATTCTTCCAAATTTTCTGATTTCAGAGAGAAG
GCCTTAATGTTTGGCCTGATTGTCGAGAAAAAGGCATCTGGAGCGTGGGT
CCTGGATTCTATCAGCCACTTCAAATGAGCTAGTCTAACTTCTAGCTTCTG
AACAATCCCCGGTTTACTCAGTCTCTCCTAATTCCAGCCTCTCGAACAACT
AATATCCTGTCTTTTCTATCCCTATGAAAAAAATTTTCATAGATTCAACTG
TTTTCATAGTAAAACCAACGTAACTAAGCTCTAACAGAGATCGATCTGTTT
CCTTGACACTATGAAGTGCCTTTTGTACTTAGCCTTTTTATTCATTGGGGTG
AATTGCATGTTTGTTTTTCTTGTTTTATTGCCACTAGTCTCTAGTCAGTGTG
TTAATCTTACAACCAGAACTCAATTACCCCCTGCATACACTAATTCTTTCA
CACGTGGTGTTTATTACCCTGACAAAGTTTTCAGATCCTCAGTTTTACATT
CAACTCAGGACTTGTTCTTACCTTTCTTTTCCAATGTTACTTGGTTCCATGC
TATACATGTCTCTGGGACCAATGGTACTAAGAGGTTTGATAACCCTGTCCT
ACCATTTAATGATGGTGTTTATTTTGCTTCCACTGAGAAGTCTAACATAAT
AAGAGGCTGGATTTTTGGTACTACTTTAGATTCGAAGACCCAGTCCCTACT
TATTGTTAATAACGCTACTAATGTTGTTATTAAAGTCTGTGAATTTCAATTT
TGTAATGATCCATTTTTGGGTGTTTATTACCACAAAAACAACAAAAGTTGG
ATGGAAAGTGAGTTCAGAGTTTATTCTAGTGCGAATAATTGCACTTTTGAA
TATGTCTCTCAGCCTTTTCTTATGGACCTTGAAGGAAAACAGGGTAATTTC
AAAAATCTTAGGGAATTTGTGTTTAAGAATATTGATGGTTATTTTAAAATA
TATTCTAAGCACACGCCTATTAATTTAGTGCGTGATCTCCCTCAGGGTTTT
TCGGCTTTAGAACCATTGGTAGATTTGCCAATAGGTATTAACATCACTAGG
TTTCAAACTTTACTTGCTTTACATAGAAGTTATTTGACTCCTGGTGATTCTT
CTTCAGGTTGGACAGCTGGTGCTGCAGCTTATTATGTGGGTTATCTTCAAC
CTAGGACTTTTCTATTAAAATATAATGAAAATGGAACCATTACAGATGCT
GTAGACTGTGCACTTGACCCTCTCTCAGAAACAAAGTGTACGTTGAAATC
CTTCACTGTAGAAAAAGGAATCTATCAAACTTCTAACTTTAGAGTCCAACC
AACAGAATCTATTGTTAGATTTCCTAATATTACAAACTTGTGCCCTTTTGG
TGAAGTTTTTAACGCCACCAGATTTGCATCTGTTTATGCTTGGAACAGGAA
GAGAATCAGCAACTGTGTTGCTGATTATTCTGTCCTATATAATTCCGCATC
ATTTTCCACTTTTAAGTGTTATGGAGTGTCTCCTACTAAATTAAATGATCTC
TGCTTTACTAATGTCTATGCAGATTCATTTGTAATTAGAGGTGATGAAGTC
AGACAAATCGCTCCAGGGCAAACTGGAAAGATTGCTGATTATAATTATAA
ATTACCAGATGATTTTACAGGCTGCGTTATAGCTTGGAATTCTAACAATCT
TGATTCTAAGGTTGGTGGTAATTATAATTACCTGTATAGATTGTTTAGGAA
GTCTAATCTCAAACCTTTTGAGAGAGATATTTCAACTGAAATCTATCAGGC
CGGTAGCACACCTTGTAATGGTGTTGAAGGTTTTAATTGTTACTTTCCTTT
ACAATCATATGGTTTCCAACCCACTAATGGTGTTGGTTACCAACCATACAG
AGTAGTAGTACTTTCTTTTGAACTTCTACATGCACCAGCAACTGTTTGTGG
ACCTAAAAAGTCTACTAATTTGGTTAAAAACAAATGTGTCAATTTCAACTT
CAATGGTTTAACAGGCACAGGTGTTCTTACTGAGTCTAACAAAAAGTTTCT
GCCTTTCCAACAATTTGGCAGAGACATTGCTGACACTACTGATGCTGTCCG
TGATCCACAGACACTTGAGATTCTTGACATTACACCATGTTCTTTTGGTGG
TGTCAGTGTTATAACACCAGGAACAAATACTTCTAACCAGGTTGCTGTTCT
TTATCAGGATGTTAACTGCACAGAAGTCCCTGTTGCTATTCATGCAGATCA
ACTTACTCCTACTTGGCGTGTTTATTCTACAGGTTCTAATGTTTTTCAAACA
CGTGCAGGCTGTTTAATAGGGGCTGAACATGTCAACAACTCATATGAGTG
TGACATACCCATTGGTGCAGGTATATGCGCTAGTTATCAGACTCAGACTA
ATTCTCCTCGGCGGGCACGTAGTGTAGCTAGTCAATCCATCATTGCCTACA
CTATGTCACTTGGTGCAGAAAATTCAGTTGCTTACTCTAATAACTCTATTG
CCATACCCACAAATTTTACTATTAGTGTTACCACAGAAATTCTACCAGTGT
CTATGACCAAGACATCAGTAGATTGTACAATGTACATTTGTGGTGATTCAA
CTGAATGCAGCAATCTTTTGTTGCAATATGGCAGTTTTTGTACACAATTAA
ACCGTGCTTTAACTGGAATAGCTGTTGAACAAGACAAAAACACCCAAGAA
GTTTTTGCACAAGTCAAACAAATTTACAAAACACCACCAATTAAAGATTTT
GGTGGTTTTAATTTTTCACAAATATTACCAGATCCATCAAAACCAAGCAAG
AGGTCATTTATTGAAGATCTACTTTTCAACAAAGTGACACTTGCAGATGCT
GGCTTCATCAAACAATATGGTGATTGCCTTGGTGATATTGCTGCTAGAGAC
CTCATTTGTGCACAAAAGTTTAACGGCCTTACTGTTTTGCCACCTTTGCTC
ACAGATGAAATGATTGCTCAATACACTTCTGCACTGTTAGCGGGTACAAT
CACTTCTGGTTGGACCTTTGGTGCAGGTGCTGCATTACAAATACCATTTGC
TATGCAAATGGCTTATAGGTTTAATGGTATTGGAGTTACACAGAATGTTCT
CTATGAGAACCAAAAATTGATTGCCAACCAATTTAATAGTGCTATTGGCA
AAATTCAAGACTCACTTTCTTCCACAGCAAGTGCACTTGGAAAACTTCAA
GATGTGGTCAACCAAAATGCACAAGCTTTAAACACGCTTGTTAAACAACT
TAGCTCCAATTTTGGTGCAATTTCAAGTGTTTTAAATGATATCCTTTCACGT
CTTGACAAAGTTGAGGCTGAAGTGCAAATTGATAGGTTGATCACAGGCAG
ACTTCAAAGTTTGCAGACATATGTGACTCAACAATTAATTAGAGCTGCAG
AAATCAGAGCTTCTGCTAATCTTGCTGCTACTAAAATGTCAGAGTGTGTAC
TTGGACAATCAAAAAGAGTTGATTTTTGTGGAAAGGGCTATCATCTTATGT
CCTTCCCTCAGTCAGCACCTCATGGTGTAGTCTTCTTGCATGTGACTTATGT
CCCTGCACAAGAAAAGAACTTCACAACTGCTCCTGCCATTTGTCATGATG
GAAAAGCACACTTTCCTCGTGAAGGTGTCTTTGTTTCAAATGGCACACACT
GGTTTGTAACACAAAGGAATTTTTATGAACCACAAATCATTACTACAGAC
AACACATTTGTGTCTGGTAACTGTGATGTTGTAATAGGAATTGTCAACAAC
ACAGTTTATGATCCTTTGCAACCTGAATTAGACTCATTCAAGGAGGAGTTA
GATAAATATTTTAAGAATCATACATCACCAGATGTTGATTTAGGTGACATC
TCTGGCATTAATGCTTCAGTTGTAAACATTCAAAAAGAAATTGACCGCCTC
AATGAGGTTGCCAAGAATTTAAATGAATCTCTCATCGATCTCCAAGAACTT
GGAAAGTATGAGCAGTATATAAAATGGCCATGGTACATTTGGCTAGGTTT
TATAGCTGGCTTGATTGCCATAGTAATGGTGACAATTATGCTTTGCTGTAT
GACCAGTTGCTGTAGTTGTCTCAAGGGCTGTTGTTCTTGTGGATCCTGCTG
CAAATTTGATGAAGACGACTCTGAGCCAGTGCTCAAAGGAGTCAAATTAC
ATTACACATAATGAAAAAAATCATCCCAATAGTGCTAATACTAATGCCGT
CAACTGTTTGCTGCATAATAAACAATACAACGTTTGCTGTGCTACATAGGC
CGTCTAGTCTAGCTAATTAACAGCAATCATGGAAGTCCACGATTTTGAGA
CCGACGAGTTCAATGATTTCAATGAAGATGACTATGCCACAAGAGAATTC
CTGAATCCCGATGAGCGCATGACGTACTTGAATCATGCTGATTACAATTTG
AATTCTCCTCTAATTAGTGATGATATTGACAATTTGATCAGGAAATTCAAT
TCTCTTCCGATTCCCTCGATGTGGGATAGTAAGAACTGGGATGGAGTTCTT
GAGATGTTAACATCATGTCAAGCCAATCCCATCTCAACATCTCAGATGCAT
AAATGGATGGGAAGTTGGTTAATGTCTGATAATCATGATGCCAGTCAAGG
GTATAGTTTTTTACATGAAGTGGACAAAGAGGCAGAAATAACATTTGACG
TGGTGGAGACCTTCATCCGCGGCTGGGGCAACAAACCAATTGAATACATC
AAAAAGGAAAGATGGACTGACTCATTCAAAATTCTCGCTTATTTGTGTCA
AAAGTTTTTGGACTTACACAAGTTGACATTAATCTTAAATGCTGTCTCTGA
GGTGGAATTGCTCAACTTGGCGAGGACTTTCAAAGGCAAAGTCAGAAGAA
GTTCTCATGGAACGAACATATGCAGGATTAGGGTTCCCAGCTTGGGTCCT
ACTTTTATTTCAGAAGGATGGGCTTACTTCAAGAAACTTGATATTCTAATG
GACCGAAACTTTCTGTTAATGGTCAAAGATGTGATTATAGGGAGGATGCA
AACGGTGCTATCCATGGTATGTAGAATAGACAACCTGTTCTCAGAGCAAG
ACATCTTCTCCCTTCTAAATATCTACAGAATTGGAGATAAAATTGTGGAGA
GGCAGGGAAATTTTTCTTATGACTTGATTAAAATGGTGGAACCGATATGC
AACTTGAAGCTGATGAAATTAGCAAGAGAATCAAGGCCTTTAGTCCCACA
ATTCCCTCATTTTGAAAATCATATCAAGACTTCTGTTGATGAAGGGGCAAA
AATTGACCGAGGTATAAGATTCCTCCATGATCAGATAATGAGTGTGAAAA
CAGTGGATCTCACACTGGTGATTTATGGATCGTTCAGACATTGGGGTCATC
CTTTTATAGATTATTACACTGGACTAGAAAAATTACATTCCCAAGTAACCA
TGAAGAAAGATATTGATGTGTCATATGCAAAAGCACTTGCAAGTGATTTA
GCTCGGATTGTTCTATTTCAACAGTTCAATGATCATAAAAAGTGGTTCGTG
AATGGAGACTTGCTCCCTCATGATCATCCCTTTAAAAGTCATGTTAAAGAA
AATACATGGCCCACAGCTGCTCAAGTTCAAGATTTTGGAGATAAATGGCA
TGAACTTCCGCTGATTAAATGTTTTGAAATACCCGACTTACTAGACCCATC
GATAATATACTCTGACAAAAGTCATTCAATGAATAGGTCAGAGGTGTTGA
AACATGTCCGAATGAATCCGAACACTCCTATCCCTAGTAAAAAGGTGTTG
CAGACTATGTTGGACACAAAGGCTACCAATTGGAAAGAATTTCTTAAAGA
GATTGATGAGAAGGGCTTAGATGATGATGATCTAATTATTGGTCTTAAAG
GAAAGGAGAGGGAACTGAAGTTGGCAGGTAGATTTTTCTCCCTAATGTCT
TGGAAATTGCGAGAATACTTTGTAATTACCGAATATTTGATAAAGACTCAT
TTCGTCCCTATGTTTAAAGGCCTGACAATGGCGGACGATCTAACTGCAGTC
ATTAAAAAGATGTTAGATTCCTCATCCGGCCAAGGATTGAAGTCATATGA
GGCAATTTGCATAGCCAATCACATTGATTACGAAAAATGGAATAACCACC
AAAGGAAGTTATCAAACGGCCCAGTGTTCCGAGTTATGGGCCAGTTCTTA
GGTTATCCATCCTTAATCGAGAGAACTCATGAATTTTTTGAGAAAAGTCTT
ATATACTACAATGGAAGACCAGACTTGATGCGTGTTCACAACAACACACT
GATCAATTCAACCTCCCAACGAGTTTGTTGGCAAGGACAAGAGGGTGGAC
TGGAAGGTCTACGGCAAAAAGGATGGACTATCCTCAATCTACTGGTTATT
CAAAGAGAGGCTAAAATCAGAAACACTGCTGTCAAAGTCTTGGCACAAG
GTGATAATCAAGTTATTTGCACACAGTATAAAACGAAGAAATCGAGAAAC
GTTGTAGAATTACAGGGTGCTCTCAATCAAATGGTTTCTAATAATGAGAA
AATTATGACTGCAATCAAAATAGGGACAGGGAAGTTAGGACTTTTGATAA
ATGACGATGAGACTATGCAATCTGCAGATTACTTGAATTATGGAAAAATA
CCGATTTTCCGTGGAGTGATTAGAGGGTTAGAGACCAAGAGATGGTCACG
AGTGACTTGTGTCACCAATGACCAAATACCCACTTGTGCTAATATAATGA
GCTCAGTTTCCACAAATGCTCTCACCGTAGCTCATTTTGCTGAGAACCCAA
TCAATGCCATGATACAGTACAATTATTTTGGGACATTTGCTAGACTCTTGT
TGATGATGCATGATCCTGCTCTTCGTCAATCATTGTATGAAGTTCAAGATA
AGATACCGGGCTTGCACAGTTCTACTTTCAAATACGCCATGTTGTATTTGG
ACCCTTCCATTGGAGGAGTGTCGGGCATGTCTTTGTCCAGGTTTTTGATTA
GAGCCTTCCCAGATCCCGTAACAGAAAGTCTCTCATTCTGGAGATTCATCC
ATGTACATGCTCGAAGTGAGCATCTGAAGGAGATGAGTGCAGTATTTGGA
AACCCCGAGATAGCCAAGTTTCGAATAACTCACATAGACAAGCTAGTAGA
AGATCCAACCTCTCTGAACATCGCTATGGGAATGAGTCCAGCGAACTTGT
TAAAGACTGAGGTTAAAAAATGCTTAATCGAATCAAGACAAACCATCAGG
AACCAGGTGATTAAGGATGCAACCATATATTTGTATCATGAAGAGGATCG
GCTCAGAAGTTTCTTATGGTCAATAAATCCTCTGTTCCCTAGATTTTTAAG
TGAATTCAAATCAGGCACTTTTTTGGGAGTCGCAGACGGGCTCATCAGTCT
ATTTCAAAATTCTCGTACTATTCGGAACTCCTTTAAGAAAAAGTATCATAG
GGAATTGGATGATTTGATTGTGAGGAGTGAGGTATCCTCTTTGACACATTT
AGGGAAACTTCATTTGAGAAGGGGATCATGTAAAATGTGGACATGTTCAG
CTACTCATGCTGACACATTAAGATACAAATCCTGGGGCCGTACAGTTATTG
GGACAACTGTACCCCATCCATTAGAAATGTTGGGTCCACAACATCGAAAA
GAGACTCCTTGTGCACCATGTAACACATCAGGGTTCAATTATGTTTCTGTG
CATTGTCCAGACGGGATCCATGACGTCTTTAGTTCACGGGGACCATTGCCT
GCTTATCTAGGGTCTAAAACATCTGAATCTACATCTATTTTGCAGCCTTGG
GAAAGGGAAAGCAAAGTCCCACTGATTAAAAGAGCTACACGTCTTAGAG
ATGCTATCTCTTGGTTTGTTGAACCCGACTCTAAACTAGCAATGACTATAC
TTTCTAACATCCACTCTTTAACAGGCGAAGAATGGACCAAAAGGCAGCAT
GGGTTCAAAAGAACAGGGTCTGCCCTTCATAGGTTTTCGACATCTCGGAT
GAGCCATGGTGGGTTCGCATCTCAGAGCACTGCAGCATTGACCAGGTTGA
TGGCAACTACAGACACCATGAGGGATCTGGGAGATCAGAATTTCGACTTT
TTATTCCAAGCAACGTTGCTCTATGCTCAAATTACCACCACTGTTGCAAGA
GACGGATGGATCACCAGTTGTACAGATCATTATCATATTGCCTGTAAGTCC
TGTTTGAGACCCATAGAAGAGATCACCCTGGACTCAAGTATGGACTACAC
GCCCCCAGATGTATCCCATGTGCTGAAGACATGGAGGAATGGGGAAGGTT
CGTGGGGACAAGAGATAAAACAGATCTATCCTTTAGAAGGGAATTGGAA
GAATTTAGCACCTGCTGAGCAATCCTATCAAGTCGGCAGATGTATAGGTTT
TCTATATGGAGACTTGGCGTATAGAAAATCTACTCATGCCGAGGACAGTT
CTCTATTTCCTCTATCTATACAAGGTCGTATTAGAGGTCGAGGTTTCTTAA
AAGGGTTGCTAGACGGATTAATGAGAGCAAGTTGCTGCCAAGTAATACAC
CGGAGAAGTCTGGCTCATTTGAAGAGGCCGGCCAACGCAGTGTACGGAGG
TTTGATTTACTTGATTGATAAATTGAGTGTATCACCTCCATTCCTTTCTCTT
ACTAGATCAGGACCTATTAGAGACGAATTAGAAACGATTCCCCACAAGAT
CCCAACCTCCTATCCGACAAGCAACCGTGATATGGGGGTGATTGTCAGAA
ATTACTTCAAATACCAATGCCGTCTAATTGAAAAGGGAAAATACAGATCA
CATTATTCACAATTATGGTTATTCTCAGATGTCTTATCCATAGACTTCATTG
GACCATTCTCTATTTCCACCACCCTCTTGCAAATCCTATACAAGCCATTTTT
ATCTGGGAAAGATAAGAATGAGTTGAGAGAGCTGGCAAATCTTTCTTCAT
TGCTAAGATCAGGAGAGGGGTGGGAAGACATACATGTGAAATTCTTCACC
AAGGACATATTATTGTGTCCAGAGGAAATCAGACATGCTTGCAAGTTCGG
GATTGCTAAGGATAATAATAAAGACATGAGCTATCCCCCTTGGGGAAGGG
AATCCAGAGGGACAATTACAACAATCCCTGTTTATTATACGACCACCCCTT
ACCCAAAGATGCTAGAGATGCCTCCAAGAATCCAAAATCCCCTGCTGTCC
GGAATCAGGTTGGGCCAATTACCAACTGGCGCTCATTATAAAATTCGGAG
TATATTACATGGAATGGGAATCCATTACAGGGACTTCTTGAGTTGTGGAG
ACGGCTCCGGAGGGATGACTGCTGCATTACTACGAGAAAATGTGCATAGC
AGAGGAATATTCAATAGTCTGTTAGAATTATCAGGGTCAGTCATGCGAGG
CGCCTCTCCTGAGCCCCCCAGTGCCCTAGAAACTTTAGGAGGAGATAAAT
CGAGATGTGTAAATGGTGAAACATGTTGGGAATATCCATCTGACTTATGT
GACCCAAGGACTTGGGACTATTTCCTCCGACTCAAAGCAGGCTTGGGGCT
TCAAATTGATTTAATTGTAATGGATATGGAAGTTCGGGATTCTTCTACTAG
CCTGAAAATTGAGACGAATGTTAGAAATTATGTGCACCGGATTTTGGATG
AGCAAGGAGTTTTAATCTACAAGACTTATGGAACATATATTTGTGAGAGC
GAAAAGAATGCAGTAACAATCCTTGGTCCCATGTTCAAGACGGTCGACTT
AGTTCAAACAGAATTTAGTAGTTCTCAAACGTCTGAAGTATATATGGTATG
TAAAGGTTTGAAGAAATTAATCGATGAACCCAATCCCGATTGGTCTTCCAT
CAATGAATCCTGGAAAAACCTGTACGCATTCCAGTCATCAGAACAGGAAT
TTGCCAGAGCAAAGAAGGTTAGTACATACTTTACCTTGACAGGTATTCCCT
CCCAATTCATTCCTGATCCTTTTGTAAACATTGAGACTATGCTACAAATAT
TCGGAGTACCCACGGGTGTGTCTCATGCGGCTGCCTTAAAATCATCTGATA
GACCTGCAGATTTATTGACCATTAGCCTTTTTTATATGGCGATTATATCGT
ATTATAACATCAATCATATCAGAGTAGGACCGATACCTCCGAACCCCCCA
TCAGATGGAATTGCACAAAATGTGGGGATCGCTATAACTGGTATAAGCTT
TTGGCTGAGTTTGATGGAGAAAGACATTCCACTATATCAACAGTGTTTAGC
AGTTATCCAGCAATCATTCCCGATTAGGTGGGAGGCTGTTTCAGTAAAAG
GAGGATACAAGCAGAAGTGGAGTACTAGAGGTGATGGGCTCCCAAAAGA
TACCCGAACTTCAGACTCCTTGGCCCCAATCGGGAACTGGATCAGATCTCT
GGAATTGGTCCGAAACCAAGTTCGTCTAAATCCATTCAATGAGATCTTGTT
CAATCAGCTATGTCGTACAGTGGATAATCATTTGAAATGGTCAAATTTGCG
AAGAAACACAGGAATGATTGAATGGATCAATAGACGAATTTCAAAAGAA
GACCGGTCTATACTGATGTTGAAGAGTGACCTACACGAGGAAAACTCTTG
GAGAGATTAAAAAATCATGAGGAGACTCCAAACTTTAAGTATG (SEQ ID
NO: 1)

An example of an artificial vesicular stomatitis virus engineered with the spike glycoprotein gene of severe acute respiratory syndrome coronavirus 2 and hemagglutinin gene of measles virus, complete genome, is identified as HGI-073.1.

| | |
|---|---|
| mRNA | 51..1376 |
| | /product = ″N mRNA″ |
| | /note = ″Nucleocapsid″ |
| | /reference = ″J02428.1″ |
| mRNA | 1386..2199 |
| | /product = ″P mRNA″ |
| | /note = ″Phosphoprotein″ |
| | /reference = ″J02428.1″ |
| mRNA | 2209..3039 |
| | /product = ″M mRNA″ |
| | /note = ″Matrix″ |
| | /reference = ″J02428.1″ |
| Variation | 3047..3094 |
| | /product = ″VA1″ |
| | /note = ″Viral Adaptor 1″ |
| | /reference = ″HGI-007.1″ |
| Variation | 3095..3096 |
| | /product = ″GB1″ |
| | /note = ″Gene Boundary 1″ |
| UTR | 3097..3125 |
| | /product = ″VSV-G UTR″ |
| | /note = ″VSV Glycoprotein UTR″ |
| | /reference = ″J02428.1″ |
| mRNA | 3126..3173 |
| | /product = ″Signal Peptide″ |
| | /note = ″VSV-G Signal peptide″ |
| | /reference = ″J02428.1″ |
| CDS | 3174..6995 |
| | /product = ″COVID-S″ |
| | /note = ″SARS-CoV-2 Spike Glycoprotein″ |
| | /reference = ″NC_045512.2″ |
| mRNA | 6996..7004 |
| | /product = ″VSV-G Stop″ |
| | /note = ″VSV-G Consensus Stop Sequence″ |
| | /reference = ″J02428.1″ |
| Variation | 7005..7046 |
| | /product = ″VA2″ |
| | /note = ″Viral Adaptor 2″ |
| | /reference = ″HGI-007.1″ |
| CDS | 7047..8988 |
| | /product = ″MV-H″ |
| | /note = ″Measles virus (Zagreb Strain)″ |
| | /reference = ″AF266290.1″ |
| Variation | 8989..9033 |
| | /product = ″VA3″ |
| | /note = ″Viral Adaptor 3″ |
| | /reference = ″HGI-007.1″ |
| Variation | 9034..9090 |
| | /product = ″VA4″ |
| | /note = ″Viral Adaptor 4″ |
| | /reference = ″HGI-007.1″ |
| mRNA | 9091..15463 |
| | /product = ″L mRNA″ |
| | /note = ″Polymerase″ |
| | /reference = ″J02428.1″ |

ACGAAGACAAACAAACCATTATTATCATTAAAAGGCTCAGG
AGAAACTTTAACAGTAATCAAAATGTCTGTTACAGTCAAGAGAATCATTG
ACAACACAGTCATAGTTCCAAAACTTCCTGCAAATGAGGATCCAGTGGAA
TACCCGGCAGATTACTTCAGAAAATCAAAGGAGATTCCTCTTTACATCAAT
ACTACAAAAAGTTTGTCAGATCTAAGAGGATATGTCTACCAAGGCCTCAA
ATCCGGAAATGTATCAATCATACATGTCAACAGCTACTTGTATGGAGCATT
AAAGGACATCCGGGGTAAGTTGGATAAAGATTGGTCAAGTTTCGGAATAA
ACATCGGGAAAGCAGGGGATACAATCGGAATATTTGACCTTGTATCCTTG
AAAGCCCTGGACGGCGTACTTCCAGATGGAGTATCGGATGCTTCCAGAAC
CAGCGCAGATGACAAATGGTTGCCTTTGTATCTACTTGGCTTATACAGAGT
GGGCAGAACACAAATGCCTGAATACAGAAAAAAGCTCATGGATGGGCTG
ACAAATCAATGCAAAATGATCAATGAACAGTTTGAACCTCTTGTGCCAGA
AGGTCGTGACATTTTTGATGTGTGGGGAAATGACAGTAATTACACAAAAA
TTGTCGCTGCAGTGGACATGTTCTTCCACATGTTCAAAAAACATGAATGTG
CCTCGTTCAGATACGGAACTATTGTTTCCAGATTCAAAGATTGTGCTGCAT
TGGCAACATTTGGACACCTCTGCAAAATAACCGGAATGTCTACAGAAGAT
GTAACGACCTGGATCTTGAACCGAGAAGTTGCAGATGAAATGGTCCAAAT
GATGCTTCCAGGCCAAGAAATTGACAAGGCCGATTCATACATGCCTTATTT
GATCGACTTTGGATTGTCTTCTAAGTCTCCATATTCTTCCGTCAAAAACCC
TGCCTTCCACTTCTGGGGGCAATTGACAGCTCTTCTGCTCAGATCCACCAG
AGCAAGGAATGCCCGACAGCCTGATGACATTGAGTATACATCTCTTACTA
CAGCAGGTTTGTTGTACGCTTATGCAGTAGGATCCTCTGCCGACTTGGCAC
AACAGTTTTGTGTTGGAGATAACAAATACACTCCAGATGATAGTACCGGA
GGATTGACGACTAATGCACCGCCACAAGGCAGAGATGTGGTCGAATGGCT
CGGATGGTTTGAAGATCAAAACAGAAAACCGACTCCTGATATGATGCAGT
ATGCGAAAAGAGCAGTCATGTCACTGCAAGGCCTAAGAGAGAAGACAAT
TGGCAAGTATGCTAAGTCAGAATTTGACAAATGACCCTATAATTCTCAGA
TCACCTATTATATATTATGCTACATATGAAAAAAACTAACAGATATCATGG
ATAATCTCACAAAAGTTCGTGAGTATCTCAAGTCCTATTCTCGTCTGGATC
AGGCGGTAGGAGAGATAGATGAGATCGAAGCACAACGAGCTGAAAAGTC
CAATTATGAGTTGTTCCAAGAGGATGGAGTGGAAGAGCATACTAAGCCCT
CTTATTTTCAGGCAGCAGATGATTCTGACACAGAATCTGAACCAGAAATT
GAAGACAATCAAGGTTTGTATGCACAGGATCCAGAAGCTGAGCAAGTTGA
AGGCTTTATACAGGGGCCTTTAGATGACTATGCAGATGAGGAAGTGGATG
TTGTATTTACTTCGGACTGGAAACCACCTGAGCTTGAATCTGACGAGCATG
GAAAGACCTTACGGTTGACATCGCCAGAGGGTTTAAGTGGAGAGCAGAA
ATCCCAGTGGCTTTCGACGATTAAAGCAGTCGTGCAAAGTGCCAAATACT
GGAATCTGGCAGAGTGCACATTTGAAGCATCGGGAGAAGGGGTCATTATG
AAGGAGCGCCAGATAACTCCGGATGTATATAAGGTCACTCCAGTGATGAA
CACACATCCGTCCCAATCAGAAGCAGTATCAGATGTTTGGTCTCTCTCAAA
GACATCCATGACTTTCCAACCCAAGAAAGCAAGTCTTCAGCCTCTCACCAT
ATCCTTGGATGAATTGTTCTCATCTAGAGGAGAGTTCATCTCTGTCGGAGG
TGACGGACGAATGTCTCATAAAGAGGCCATCCTGCTCGGCCTGAGATACA
AAAAGTTGTACAATCAGGCGAGAGTCAAATATTCTCTGTAGACTATGAAA
AAAAGTAACAGATATCACGATCTAAGTGTTATCCCAATCCATTCATCATG
AGTTCCTTAAAGAAGATTCTCGGTCTGAAGGGGAAAGGTAAGAAATCTAA
GAAATTAGGGATCGCACCACCCCCTTATGAAGAGGACACTAGCATGGAGT
ATGCTCCGAGCGCTCCAATTGACAAATCCTATTTTGGAGTTGACGAGATG
GACACCTATGATCCGAATCAATTAAGATATGAGAAATTCTTCTTTACAGTG
AAAATGACGGTTAGATCTAATCGTCCGTTCAGAACATACTCAGATGTGGC
AGCCGCTGTATCCCATTGGGATCACATGTACATCGGAATGGCAGGGAAAC
GTCCCTTCTACAAAATCTTGGCTTTTTTGGGTTCTTCTAATCTAAAGGCCAC
TCCAGCGGTATTGGCAGATCAAGGTCAACCAGAGTATCACACTCACTGCG
AAGGCAGGGCTTATTTGCCACATAGGATGGGGAAGACCCCTCCCATGCTC
AATGTACCAGAGCACTTCAGAAGACCATTCAATATAGGTCTTTACAAGGG
AACGATTGAGCTCACAATGACCATCTACGATGATGAGTCACTGGAAGCAG
CTCCTATGATCTGGGATCATTTCAATTCTTCCAAATTTTCTGATTTCAGAGA
GAAGGCCTTAATGTTTGGCCTGATTGTCGAGAAAAAGGCATCTGGAGCGT
GGGTCCTGGATTCTATCAGCCACTTCAAATGAGCTAGTCTAACTTCTAGCT
TCTGAACAATCCCCGGTTTACTCAGTCTCTCCTAATTCCAGCCTCTCGAAC
AACTAATATCCTGTCTTTTCTATCCCTATGAAAAAAATTTTCATAGATTCA
ACTGTTTTCATAGTAAAACCAACGTAACTAAGCTTTTTCATAGATTCAACT
GTTTTCATAGTAAAACCAACGTAACTAAGCTCTAACAGAGATCGATCTGTT
TCCTTGACACTATGAAGTGCCTTTTGTACTTAGCCTTTTTATTCATTGGGGT
GAATTGCATGTTTGTTTTTCTTGTTTTATTGCCACTAGTCTCTAGTCAGTGT
GTTAATCTTACAACCAGAACTCAATTACCCCCTGCATACACTAATTCTTTC
ACACGTGGTGTTTATTACCCTGACAAAGTTTTCAGATCCTCAGTTTTACAT
TCAACTCAGGACTTGTTCTTACCTTTCTTTTCCAATGTTACTTGGTTCCATG
CTATACATGTCTCTGGGACCAATGGTACTAAGAGGTTTGATAACCCTGTCC
TACCATTTAATGATGGTGTTTATTTTGCTTCCACTGAGAAGTCTAACATAA
TAAGAGGCTGGATTTTTGGTACTACTTTAGATTCGAAGACCCAGTCCCTAC
TTATTGTTAATAACGCTACTAATGTTGTTATTAAAGTCTGTGAATTTCAATT
TTGTAATGATCCATTTTTGGGTGTTTATTACCACAAAAACAACAAAAGTTG
GATGGAAAGTGAGTTCAGAGTTTATTCTAGTGCGAATAATTGCACTTTTGA
ATATGTCTCTCAGCCTTTTCTTATGGACCTTGAAGGAAAACAGGGTAATTT
CAAAAATCTTAGGGAATTTGTGTTTAAGAATATTGATGGTTATTTTAAAAT
ATATTCTAAGCACACGCCTATTAATTTAGTGCGTGATCTCCCTCAGGGTTT
TTCGGCTTTAGAACCATTGGTAGATTTGCCAATAGGTATTAACATCACTAG
GTTTCAAACTTTACTTGCTTTACATAGAAGTTATTTGACTCCTGGTGATTCT
TCTTCAGGTTGGACAGCTGGTGCTGCAGCTTATTATGTGGGTTATCTTCAA
CCTAGGACTTTTCTATTAAAATATAATGAAAATGGAACCATTACAGATGCT
GTAGACTGTGCACTTGACCCTCTCTCAGAAACAAAGTGTACGTTGAAATC
CTTCACTGTAGAAAAAGGAATCTATCAAACTTCTAACTTTAGAGTCCAACC
AACAGAATCTATTGTTAGATTTCCTAATATTACAAACTTGTGCCCTTTTGG
TGAAGTTTTTAACGCCACCAGATTTGCATCTGTTTATGCTTGGAACAGGAA
GAGAATCAGCAACTGTGTTGCTGATTATTCTGTCCTATATAATTCCGCATC
ATTTTCCACTTTTAAGTGTTATGGAGTGTCTCCTACTAAATTAAATGATCTC
TGCTTTACTAATGTCTATGCAGATTCATTTGTAATTAGAGGTGATGAAGTC
AGACAAATCGCTCCAGGGCAAACTGGAAAGATTGCTGATTATAATTATAA
ATTACCAGATGATTTTACAGGCTGCGTTATAGCTTGGAATTCTAACAATCT
TGATTCTAAGGTTGGTGGTAATTATAATTACCTGTATAGATTGTTTAGGAA
GTCTAATCTCAAACCTTTTGAGAGAGATATTTCAACTGAAATCTATCAGGC
CGGTAGCACACCTTGTAATGGTGTTGAAGGTTTTAATTGTTACTTTCCTTT
ACAATCATATGGTTTCCAACCCACTAATGGTGTTGGTTACCAACCATACAG
AGTAGTAGTACTTTCTTTTGAACTTCTACATGCACCAGCAACTGTTTGTGG
ACCTAAAAAGTCTACTAATTTGGTTAAAAACAAATGTGTCAATTTCAACTT
CAATGGTTTAACAGGCACAGGTGTTCTTACTGAGTCTAACAAAAAGTTTCT
GCCTTTCCAACAATTTGGCAGAGACATTGCTGACACTACTGATGCTGTCCG
TGATCCACAGACACTTGAGATTCTTGACATTACACCATGTTCTTTTGGTGG
TGTCAGTGTTATAACACCAGGAACAAATACTTCTAACCAGGTTGCTGTTCT
TTATCAGGATGTTAACTGCACAGAAGTCCCTGTTGCTATTCATGCAGATCA
ACTTACTCCTACTTGGCGTGTTTATTCTACAGGTTCTAATGTTTTTCAAACA
CGTGCAGGCTGTTTAATAGGGGCTGAACATGTCAACAACTCATATGAGTG
TGACATACCCATTGGTGCAGGTATATGCGCTAGTTATCAGACTCAGACTA
ATTCTCCTCGGCGGGCACGTAGTGTAGCTAGTCAATCCATCATTGCCTACA
CTATGTCACTTGGTGCAGAAAATTCAGTTGCTTACTCTAATAACTCTATTG
CCATACCCACAAATTTTACTATTAGTGTTACCACAGAAATTCTACCAGTGT
CTATGACCAAGACATCAGTAGATTGTACAATGTACATTTGTGGTGATTCAA
CTGAATGCAGCAATCTTTTGTTGCAATATGGCAGTTTTTGTACACAATTAA
ACCGTGCTTTAACTGGAATAGCTGTTGAACAAGACAAAAACACCCAAGAA
GTTTTTGCACAAGTCAAACAAATTTACAAAACACCACCAATTAAAGATTTT
GGTGGTTTTAATTTTTCACAAATATTACCAGATCCATCAAAACCAAGCAAG
AGGTCATTTATTGAAGATCTACTTTTCAACAAAGTGACACTTGCAGATGCT
GGCTTCATCAAACAATATGGTGATTGCCTTGGTGATATTGCTGCTAGAGAC
CTCATTTGTGCACAAAAGTTTAACGGCCTTACTGTTTTGCCACCTTTGCTC
ACAGATGAAATGATTGCTCAATACACTTCTGCACTGTTAGCGGGTACAAT
CACTTCTGGTTGGACCTTTGGTGCAGGTGCTGCATTACAAATACCATTTGC
TATGCAAATGGCTTATAGGTTTAATGGTATTGGAGTTACACAGAATGTTCT
CTATGAGAACCAAAAATTGATTGCCAACCAATTTAATAGTGCTATTGGCA
AAATTCAAGACTCACTTTCTTCCACAGCAAGTGCACTTGGAAAACTTCAA
GATGTGGTCAACCAAAATGCACAAGCTTTAAACACGCTTGTTAAACAACT
TAGCTCCAATTTTGGTGCAATTTCAAGTGTTTTAAATGATATCCTTTCACGT
CTTGACAAAGTTGAGGCTGAAGTGCAAATTGATAGGTTGATCACAGGCAG
ACTTCAAAGTTTGCAGACATATGTGACTCAACAATTAATTAGAGCTGCAG
AAATCAGAGCTTCTGCTAATCTTGCTGCTACTAAAATGTCAGAGTGTGTAC
TTGGACAATCAAAAAGAGTTGATTTTTGTGGAAAGGGCTATCATCTTATGT
CCTTCCCTCAGTCAGCACCTCATGGTGTAGTCTTCTTGCATGTGACTTATGT
CCCTGCACAAGAAAAGAACTTCACAACTGCTCCTGCCATTTGTCATGATG
GAAAAGCACACTTTCCTCGTGAAGGTGTCTTTGTTTCAAATGGCACACACT
GGTTTGTAACACAAAGGAATTTTTATGAACCACAAATCATTACTACAGAC
AACACATTTGTGTCTGGTAACTGTGATGTTGTAATAGGAATTGTCAACAAC
ACAGTTTATGATCCTTTGCAACCTGAATTAGACTCATTCAAGGAGGAGTTA
GATAAATATTTTAAGAATCATACATCACCAGATGTTGATTTAGGTGACATC
TCTGGCATTAATGCTTCAGTTGTAAACATTCAAAAAGAAATTGACCGCCTC
AATGAGGTTGCCAAGAATTTAAATGAATCTCTCATCGATCTCCAAGAACTT
GGAAAGTATGAGCAGTATATAAAATGGCCATGGTACATTTGGCTAGGTTT
TATAGCTGGCTTGATTGCCATAGTAATGGTGACAATTATGCTTTGCTGTAT
GACCAGTTGCTGTAGTTGTCTCAAGGGCTGTTGTTCTTGTGGATCCTGCTG
CAAATTTGATGAAGACGACTCTGAGCCAGTGCTCAAAGGAGTCAAATTAC
ATTACACATAATGAAAAAAATCATCCCAATAGTGCTAATACTAATGCCGT
CAACTGTTTGCTCTAACAGAGATCGATCTGTTTCCTTGACACTATGAAGTG
CCTTTTGTACTTAGCCTTTTTATTCATTGGGGTGAATTGCATGTCACCACAA
CGAGACCGGATAAATGCCTTCTACAAAGATAACCCCCATCCCAAGGGAAG
TAGGATAGTCATTAACAGAGAACATCTTATGATTGATAGACCTTATGTTTT
GCTGGCTGTTCTGTTTGTCATGTTTCTGAGCTTGATCGGGTTGCTAGCCATT
GCAGGCATTAGACTTCATCGGGCAGCCATCTACACCGCAGAGATCCATAA
AAGCCTCAGCACCAATCTAGATGTAACTAACTCAATCGAGCATCAGGTCA
AGGACGTGCTGACACCACTCTTCAAAATCATCGGTGATGAAGTGGGCCTG
AGGACACCTCAGAGATTCACTGACCTAGTGAAATTCATCTCTGACAAGAT
TAAATTCCTTAATCCGGATAGGGAGTACGACTTCAGAGATCTCACTTGGTG
TATCAACCCGCCAGAGAGAATCAAATTGGATTATGATCAATACTGTGCAG
ATGTGGCTGCTGAAGAGCTCATGAATGCATTGGTGAACTCAACTCTACTG
GAGACCAGAACAACCAATCAGTTCCTAGCTGTCTCAAAGGGAAACTGCTC
AGGGCCCACTACAATCAGAGGTCAATTCTCAAACATGTCGCTGTCCCTGTT
AGACTTGTATTTAGGTCGAGGTTACAATGTGTCATCTATAGTCACTATGAC
ATCCCAGGGAATGTATGGGGGAACTTACCTAGTGGAAAAGCCTAATCTGA
GCAGCAAAAGGTCAGAGTTGTCACAACTGAGCATGTACCGAGTGTTTGAA
GTAGGTGTTATCAGAAATCCGGGTTTGGGGGCTCCGGTGTTCCATATGAC
AAACTATCTTGAGCAACCAGCCAGTAATGATCTCAGCAACTGTATGGTGG
CTTTGGGGGAGCTCAAACTCGCAGCCCTTTGTCACGGGGAAGATTCTATC
ACAATTCCCTATCAGGGATCAGGGAAAGGTGTCAGCTTCCAGCTCGTCAA
GCTAGGTGTCTGGAAATCCCCAACCGACATGCAATCCTGGGTCCCCTTATC
AACGGATGATCCAGTGATAGACAGGCTTTACCTCTCATCTCACAGAGGTG
TTATCGCTGACAATCAAGCAAAATGGGCTGTCCCGACAACACGAACAGAT
GACAAGTTGCGAATGGAGACATGCTTCCAACAGGCGTGTAAGGGTAAAAT
CCAAGCACTCTGCGAGAATCCCGAGTGGGCACCATTGAAGGATAACAGGA
TTCCTTCATACGGGGTCTTGTCTGTTGATCTGAGTCTGACAGTTGAGCTTA
AAATCAAAATTGCTTCGGGATTCGGGCCATTGATCACACACGGTTCAGGG
ATGGACCTATACAAATCCAACCACAACAATGTGTATTGGCTGACTATCCC
GCCAATGAAGAACCTAGCCTTAGGTGTAATCAACACATTGGAGTGGATAC
CGAGATTCAAGGTTAGTCCCTACCTCTTCAATGTCCCAATTAAGGAAGCA
GGCGAAGACTGCCATGCCCCAACATACCTACCTGCGGAGGTGGATGGTGA
TGTCAAACTCAGTTCCAATCTGGTGATTCTACCTGGTCAAGATCTCCAATA
TGTTTTGGCAACCTACGATACTTCCAGGGTTGAACATGCTGTGGTTTATTA
CGTTTACAGCCCAGGCCGCTCATTTTCTTACTTTTATCCTTTTAGGTTGCCT
ATAAAGGGGGTCCCCATCGAATTACAAGTGGAATGCTTCACATGGGACCA
AAAACTCTGGTGCCGTCACTTCTGTGTGCTTGCGGACTCAGAATCTGGTGG
ACATATCACTCACTCTGGGATGGTGGGCATGGGAGTCAGCTGCACAGTCA
CCCGGGAAGATGGAACCAATCGCAGATAGTGAAAAAAATTTTCCCTATAA
ATAGGCCGTCTAATATTTTAGTGCTTTTAACTAGCATAATAAACAATACAA
CGTTTGCTGTGCTACATAGGCCGTCTAGTCTAGCTAATTAACAGCAATCAT
GGAAGTCCACGATTTTGAGACCGACGAGTTCAATGATTTCAATGAAGATG
ACTATGCCACAAGAGAATTCCTGAATCCCGATGAGCGCATGACGTACTTG
AATCATGCTGATTACAATTTGAATTCTCCTCTAATTAGTGATGATATTGAC
AATTTGATCAGGAAATTCAATTCTCTTCCGATTCCCTCGATGTGGGATAGT
AAGAACTGGGATGGAGTTCTTGAGATGTTAACATCATGTCAAGCCAATCC
CATCTCAACATCTCAGATGCATAAATGGATGGGAAGTTGGTTAATGTCTG
ATAATCATGATGCCAGTCAAGGGTATAGTTTTTTACATGAAGTGGACAAA
GAGGCAGAAATAACATTTGACGTGGTGGAGACCTTCATCCGCGGCTGGGG
CAACAAACCAATTGAATACATCAAAAAGGAAAGATGGACTGACTCATTCA
AAATTCTCGCTTATTTGTGTCAAAAGTTTTTGGACTTACACAAGTTGACAT
TAATCTTAAATGCTGTCTCTGAGGTGGAATTGCTCAACTTGGCGAGGACTT
TCAAAGGCAAAGTCAGAAGAAGTTCTCATGGAACGAACATATGCAGGATT
AGGGTTCCCAGCTTGGGTCCTACTTTTATTTCAGAAGGATGGGCTTACTTC
AAGAAACTTGATATTCTAATGGACCGAAACTTTCTGTTAATGGTCAAAGA
TGTGATTATAGGGAGGATGCAAACGGTGCTATCCATGGTATGTAGAATAG
ACAACCTGTTCTCAGAGCAAGACATCTTCTCCCTTCTAAATATCTACAGAA
TTGGAGATAAAATTGTGGAGAGGCAGGGAAATTTTTCTTATGACTTGATT
AAAATGGTGGAACCGATATGCAACTTGAAGCTGATGAAATTAGCAAGAG
AATCAAGGCCTTTAGTCCCACAATTCCCTCATTTTGAAAATCATATCAAGA
CTTCTGTTGATGAAGGGGCAAAAATTGACCGAGGTATAAGATTCCTCCAT
GATCAGATAATGAGTGTGAAAACAGTGGATCTCACACTGGTGATTTATGG
ATCGTTCAGACATTGGGGTCATCCTTTTATAGATTATTACACTGGACTAGA
AAAATTACATTCCCAAGTAACCATGAAGAAAGATATTGATGTGTCATATG
CAAAAGCACTTGCAAGTGATTTAGCTCGGATTGTTCTATTTCAACAGTTCA
ATGATCATAAAAAGTGGTTCGTGAATGGAGACTTGCTCCCTCATGATCATC
CCTTTAAAAGTCATGTTAAAGAAAATACATGGCCCACAGCTGCTCAAGTT
CAAGATTTTGGAGATAAATGGCATGAACTTCCGCTGATTAAATGTTTTGAA
ATACCCGACTTACTAGACCCATCGATAATATACTCTGACAAAAGTCATTCA
ATGAATAGGTCAGAGGTGTTGAAACATGTCCGAATGAATCCGAACACTCC
TATCCCTAGTAAAAAGGTGTTGCAGACTATGTTGGACACAAAGGCTACCA
ATTGGAAAGAATTTCTTAAAGAGATTGATGAGAAGGGCTTAGATGATGAT
GATCTAATTATTGGTCTTAAAGGAAAGGAGAGGGAACTGAAGTTGGCAGG
TAGATTTTTCTCCCTAATGTCTTGGAAATTGCGAGAATACTTTGTAATTAC
CGAATATTTGATAAAGACTCATTTCGTCCCTATGTTTAAAGGCCTGACAAT
GGCGGACGATCTAACTGCAGTCATTAAAAAGATGTTAGATTCCTCATCCG
GCCAAGGATTGAAGTCATATGAGGCAATTTGCATAGCCAATCACATTGAT
TACGAAAAATGGAATAACCACCAAAGGAAGTTATCAAACGGCCCAGTGTT
CCGAGTTATGGGCCAGTTCTTAGGTTATCCATCCTTAATCGAGAGAACTCA
TGAATTTTTTGAGAAAAGTCTTATATACTACAATGGAAGACCAGACTTGAT
GCGTGTTCACAACAACACACTGATCAATTCAACCTCCCAACGAGTTTGTTG
GCAAGGACAAGAGGGTGGACTGGAAGGTCTACGGCAAAAAGGATGGACT
ATCCTCAATCTACTGGTTATTCAAAGAGAGGCTAAAATCAGAAACACTGC
TGTCAAAGTCTTGGCACAAGGTGATAATCAAGTTATTTGCACACAGTATA
AAACGAAGAAATCGAGAAACGTTGTAGAATTACAGGGTGCTCTCAATCAA
ATGGTTTCTAATAATGAGAAAATTATGACTGCAATCAAAATAGGGACAGG
GAAGTTAGGACTTTTGATAAATGACGATGAGACTATGCAATCTGCAGATT
ACTTGAATTATGGAAAAATACCGATTTTCCGTGGAGTGATTAGAGGGTTA
GAGACCAAGAGATGGTCACGAGTGACTTGTGTCACCAATGACCAAATACC
CACTTGTGCTAATATAATGAGCTCAGTTTCCACAAATGCTCTCACCGTAGC
TCATTTTGCTGAGAACCCAATCAATGCCATGATACAGTACAATTATTTTGG
GACATTTGCTAGACTCTTGTTGATGATGCATGATCCTGCTCTTCGTCAATC
ATTGTATGAAGTTCAAGATAAGATACCGGGCTTGCACAGTTCTACTTTCAA
ATACGCCATGTTGTATTTGGACCCTTCCATTGGAGGAGTGTCGGGCATGTC
TTTGTCCAGGTTTTTGATTAGAGCCTTCCCAGATCCCGTAACAGAAAGTCT
CTCATTCTGGAGATTCATCCATGTACATGCTCGAAGTGAGCATCTGAAGG
AGATGAGTGCAGTATTTGGAAACCCCGAGATAGCCAAGTTTCGAATAACT
CACATAGACAAGCTAGTAGAAGATCCAACCTCTCTGAACATCGCTATGGG
AATGAGTCCAGCGAACTTGTTAAAGACTGAGGTTAAAAAATGCTTAATCG
AATCAAGACAAACCATCAGGAACCAGGTGATTAAGGATGCAACCATATAT
TTGTATCATGAAGAGGATCGGCTCAGAAGTTTCTTATGGTCAATAAATCCT
CTGTTCCCTAGATTTTTAAGTGAATTCAAATCAGGCACTTTTTTGGGAGTC
GCAGACGGGCTCATCAGTCTATTTCAAAATTCTCGTACTATTCGGAACTCC
TTTAAGAAAAAGTATCATAGGGAATTGGATGATTTGATTGTGAGGAGTGA
GGTATCCTCTTTGACACATTTAGGGAAACTTCATTTGAGAAGGGGATCATG
TAAAATGTGGACATGTTCAGCTACTCATGCTGACACATTAAGATACAAAT
CCTGGGGCCGTACAGTTATTGGGACAACTGTACCCCATCCATTAGAAATG
TTGGGTCCACAACATCGAAAAGAGACTCCTTGTGCACCATGTAACACATC
AGGGTTCAATTATGTTTCTGTGCATTGTCCAGACGGGATCCATGACGTCTT
TAGTTCACGGGGACCATTGCCTGCTTATCTAGGGTCTAAAACATCTGAATC
TACATCTATTTTGCAGCCTTGGGAAAGGGAAAGCAAAGTCCCACTGATTA
AAAGAGCTACACGTCTTAGAGATGCTATCTCTTGGTTTGTTGAACCCGACT
CTAAACTAGCAATGACTATACTTTCTAACATCCACTCTTTAACAGGCGAAG
AATGGACCAAAAGGCAGCATGGGTTCAAAAGAACAGGGTCTGCCCTTCAT
AGGTTTTCGACATCTCGGATGAGCCATGGTGGGTTCGCATCTCAGAGCACT
GCAGCATTGACCAGGTTGATGGCAACTACAGACACCATGAGGGATCTGGG
AGATCAGAATTTCGACTTTTTATTCCAAGCAACGTTGCTCTATGCTCAAAT
TACCACCACTGTTGCAAGAGACGGATGGATCACCAGTTGTACAGATCATT
ATCATATTGCCTGTAAGTCCTGTTTGAGACCCATAGAAGAGATCACCCTGG
ACTCAAGTATGGACTACACGCCCCCAGATGTATCCCATGTGCTGAAGACA
TGGAGGAATGGGGAAGGTTCGTGGGGACAAGAGATAAAACAGATCTATC
CTTTAGAAGGGAATTGGAAGAATTTAGCACCTGCTGAGCAATCCTATCAA
GTCGGCAGATGTATAGGTTTTCTATATGGAGACTTGGCGTATAGAAAATCT
ACTCATGCCGAGGACAGTTCTCTATTTCCTCTATCTATACAAGGTCGTATT
AGAGGTCGAGGTTTCTTAAAAGGGTTGCTAGACGGATTAATGAGAGCAAG
TTGCTGCCAAGTAATACACCGGAGAAGTCTGGCTCATTTGAAGAGGCCGG
CCAACGCAGTGTACGGAGGTTTGATTTACTTGATTGATAAATTGAGTGTAT
CACCTCCATTCCTTTCTCTTACTAGATCAGGACCTATTAGAGACGAATTAG
AAACGATTCCCCACAAGATCCCAACCTCCTATCCGACAAGCAACCGTGAT
ATGGGGGTGATTGTCAGAAATTACTTCAAATACCAATGCCGTCTAATTGA
AAAGGGAAAATACAGATCACATTATTCACAATTATGGTTATTCTCAGATG
TCTTATCCATAGACTTCATTGGACCATTCTCTATTTCCACCACCCTCTTGCA
AATCCTATACAAGCCATTTTTATCTGGGAAAGATAAGAATGAGTTGAGAG
AGCTGGCAAATCTTTCTTCATTGCTAAGATCAGGAGAGGGGTGGGAAGAC
ATACATGTGAAATTCTTCACCAAGGACATATTATTGTGTCCAGAGGAAAT
CAGACATGCTTGCAAGTTCGGGATTGCTAAGGATAATAATAAAGACATGA
GCTATCCCCCTTGGGGAAGGGAATCCAGAGGGACAATTACAACAATCCCT
GTTTATTATACGACCACCCCTTACCCAAAGATGCTAGAGATGCCTCCAAG
AATCCAAAATCCCCTGCTGTCCGGAATCAGGTTGGGCCAATTACCAACTG
GCGCTCATTATAAAATTCGGAGTATATTACATGGAATGGGAATCCATTAC
AGGGACTTCTTGAGTTGTGGAGACGGCTCCGGAGGGATGACTGCTGCATT
ACTACGAGAAAATGTGCATAGCAGAGGAATATTCAATAGTCTGTTAGAAT
TATCAGGGTCAGTCATGCGAGGCGCCTCTCCTGAGCCCCCCAGTGCCCTA
GAAACTTTAGGAGGAGATAAATCGAGATGTGTAAATGGTGAAACATGTTG
GGAATATCCATCTGACTTATGTGACCCAAGGACTTGGGACTATTTCCTCCG
ACTCAAAGCAGGCTTGGGGCTTCAAATTGATTTAATTGTAATGGATATGG
AAGTTCGGGATTCTTCTACTAGCCTGAAAATTGAGACGAATGTTAGAAAT
TATGTGCACCGGATTTTGGATGAGCAAGGAGTTTTAATCTACAAGACTTAT
GGAACATATATTTGTGAGAGCGAAAAGAATGCAGTAACAATCCTTGGTCC
CATGTTCAAGACGGTCGACTTAGTTCAAACAGAATTTAGTAGTTCTCAAAC
GTCTGAAGTATATATGGTATGTAAAGGTTTGAAGAAATTAATCGATGAAC
CCAATCCCGATTGGTCTTCCATCAATGAATCCTGGAAAAACCTGTACGCAT
TCCAGTCATCAGAACAGGAATTTGCCAGAGCAAAGAAGGTTAGTACATAC
TTTACCTTGACAGGTATTCCCTCCCAATTCATTCCTGATCCTTTTGTAAACA
TTGAGACTATGCTACAAATATTCGGAGTACCCACGGGTGTGTCTCATGCG
GCTGCCTTAAAATCATCTGATAGACCTGCAGATTTATTGACCATTAGCCTT
TTTTATATGGCGATTATATCGTATTATAACATCAATCATATCAGAGTAGGA
CCGATACCTCCGAACCCCCCATCAGATGGAATTGCACAAAATGTGGGGAT
CGCTATAACTGGTATAAGCTTTTGGCTGAGTTTGATGGAGAAAGACATTCC
ACTATATCAACAGTGTTTAGCAGTTATCCAGCAATCATTCCCGATTAGGTG
GGAGGCTGTTTCAGTAAAAGGAGGATACAAGCAGAAGTGGAGTACTAGA
GGTGATGGGCTCCCAAAAGATACCCGAACTTCAGACTCCTTGGCCCCAAT
CGGGAACTGGATCAGATCTCTGGAATTGGTCCGAAACCAAGTTCGTCTAA
ATCCATTCAATGAGATCTTGTTCAATCAGCTATGTCGTACAGTGGATAATC
ATTTGAAATGGTCAAATTTGCGAAGAAACACAGGAATGATTGAATGGATC
AATAGACGAATTTCAAAAGAAGACCGGTCTATACTGATGTTGAAGAGTGA
CCTACACGAGGAAAACTCTTGGAGAGATTAAAAAATCATGAGGAGACTCC
AAACTTTAAGTATG (SEQ ID NO: 2)

An example of an artificial vesicular stomatitis virus engineered with the spike glycoprotein and nucleocapsid phosphoprotein genes of severe acute respiratory syndrome coronavirus 2, complete genome, and is identified as HGI-074.1.

| | |
|---|---|
| mRNA | 51..1376 |
| | /product = ″N mRNA″ |
| | /note = ″Nucleocapsid″ |
| | /reference = ″J02428.1″ |
| mRNA | 1386..2199 |
| | /product = ″P mRNA″ |
| | /note = ″Phosphoprotein″ |
| | /reference = ″J02428.1″ |
| mRNA | 2209..3039 |
| | /product = ″M mRNA″ |
| | /note = ″Matrix″ |
| | /reference = ″J02428.1″ |
| Variation | 3047..3094 |
| | /product = ″VAl″ |
| | /note = ″Viral Adaptor 1″ |
| | /reference = ″HGI-007.1″ |
| Variation | 3095..3096 |
| | /product = ″GB1″ |
| | /note = ″Gene Boundary 1″ |
| UTR | 3097..3125 |
| | /product = ″VSV-G UTR″ |
| | /note = ″VSV Glycoprotein UTR″ |
| | /reference = ″J02428.1″ |
| mRNA | 3126..3173 |
| | /product = ″Signal Peptide″ |
| | /note = ″VSV-G Signal peptide″ |
| | /reference = ″J02428.1″ |
| CDS | 3174..6995 |
| | /product = ″COVID-S″ |
| | /note = ″SARS-CoV-2 Spike Glycoprotein″ |
| | /reference = ″NC 045512.2″ |
| mRNA | 6996..7004 |
| | /product = ″VSV-G Stop″ |
| | /note = ″VSV-G Consensus Stop Sequence″ |
| | /reference = ″J02428.1″ |
| Variation | 7005..7046 |
| | /product = ″VA2″ |
| | /note = ″Viral Adaptor 2″ |
| | /reference = ″HGI-007.1″ |
| CDS | 7047..8306 |
| | /product = ″COV-N″ |
| | /note = ″ SARS-CoV-2 Nucleoprotein″ |
| | /reference = ″NC_045512.2″ |
| Variation | 8307..8351 |
| | /product = ″VA3″ |
| | /note = ″Viral Adaptor 3″ |
| | /reference = ″HGI-007.1″ |
| Variation | 8352..8408 |
| | /product = ″VA4″ |
| | /note = ″Viral Adaptor 4″ |
| | /reference = ″HGI-007.1″ |
| mRNA | 8409..14781 |
| | /product = ″L mRNA″ |
| | /note = ″Polymerase″ |
| | /reference = ″J02428.1″ |

ACGAAGACAAACAAACCATTATTATCATTAAAAGGCTCAGG
AGAAACTTTAACAGTAATCAAAATGTCTGTTACAGTCAAGAGAATCATTG
ACAACACAGTCATAGTTCCAAAACTTCCTGCAAATGAGGATCCAGTGGAA
TACCCGGCAGATTACTTCAGAAAATCAAAGGAGATTCCTCTTTACATCAAT
ACTACAAAAAGTTTGTCAGATCTAAGAGGATATGTCTACCAAGGCCTCAA
ATCCGGAAATGTATCAATCATACATGTCAACAGCTACTTGTATGGAGCATT
AAAGGACATCCGGGGTAAGTTGGATAAAGATTGGTCAAGTTTCGGAATAA
ACATCGGGAAAGCAGGGGATACAATCGGAATATTTGACCTTGTATCCTTG
AAAGCCCTGGACGGCGTACTTCCAGATGGAGTATCGGATGCTTCCAGAAC
CAGCGCAGATGACAAATGGTTGCCTTTGTATCTACTTGGCTTATACAGAGT
GGGCAGAACACAAATGCCTGAATACAGAAAAAAGCTCATGGATGGGCTG
ACAAATCAATGCAAAATGATCAATGAACAGTTTGAACCTCTTGTGCCAGA
AGGTCGTGACATTTTTGATGTGTGGGGAAATGACAGTAATTACACAAAAA
TTGTCGCTGCAGTGGACATGTTCTTCCACATGTTCAAAAAACATGAATGTG
CCTCGTTCAGATACGGAACTATTGTTTCCAGATTCAAAGATTGTGCTGCAT
TGGCAACATTTGGACACCTCTGCAAAATAACCGGAATGTCTACAGAAGAT
GTAACGACCTGGATCTTGAACCGAGAAGTTGCAGATGAAATGGTCCAAAT
GATGCTTCCAGGCCAAGAAATTGACAAGGCCGATTCATACATGCCTTATTT
GATCGACTTTGGATTGTCTTCTAAGTCTCCATATTCTTCCGTCAAAAACCC
TGCCTTCCACTTCTGGGGGCAATTGACAGCTCTTCTGCTCAGATCCACCAG
AGCAAGGAATGCCCGACAGCCTGATGACATTGAGTATACATCTCTTACTA
CAGCAGGTTTGTTGTACGCTTATGCAGTAGGATCCTCTGCCGACTTGGCAC
AACAGTTTTGTGTTGGAGATAACAAATACACTCCAGATGATAGTACCGGA
GGATTGACGACTAATGCACCGCCACAAGGCAGAGATGTGGTCGAATGGCT
CGGATGGTTTGAAGATCAAAACAGAAAACCGACTCCTGATATGATGCAGT
ATGCGAAAAGAGCAGTCATGTCACTGCAAGGCCTAAGAGAGAAGACAAT
TGGCAAGTATGCTAAGTCAGAATTTGACAAATGACCCTATAATTCTCAGA
TCACCTATTATATATTATGCTACATATGAAAAAAACTAACAGATATCATGG
ATAATCTCACAAAAGTTCGTGAGTATCTCAAGTCCTATTCTCGTCTGGATC
AGGCGGTAGGAGAGATAGATGAGATCGAAGCACAACGAGCTGAAAAGTC
CAATTATGAGTTGTTCCAAGAGGATGGAGTGGAAGAGCATACTAAGCCCT
CTTATTTTCAGGCAGCAGATGATTCTGACACAGAATCTGAACCAGAAATT
GAAGACAATCAAGGTTTGTATGCACAGGATCCAGAAGCTGAGCAAGTTGA
AGGCTTTATACAGGGGCCTTTAGATGACTATGCAGATGAGGAAGTGGATG
TTGTATTTACTTCGGACTGGAAACCACCTGAGCTTGAATCTGACGAGCATG
GAAAGACCTTACGGTTGACATCGCCAGAGGGTTTAAGTGGAGAGCAGAA
ATCCCAGTGGCTTTCGACGATTAAAGCAGTCGTGCAAAGTGCCAAATACT
GGAATCTGGCAGAGTGCACATTTGAAGCATCGGGAGAAGGGGTCATTATG
AAGGAGCGCCAGATAACTCCGGATGTATATAAGGTCACTCCAGTGATGAA
CACACATCCGTCCCAATCAGAAGCAGTATCAGATGTTTGGTCTCTCTCAAA
GACATCCATGACTTTCCAACCCAAGAAAGCAAGTCTTCAGCCTCTCACCAT
ATCCTTGGATGAATTGTTCTCATCTAGAGGAGAGTTCATCTCTGTCGGAGG
TGACGGACGAATGTCTCATAAAGAGGCCATCCTGCTCGGCCTGAGATACA
AAAAGTTGTACAATCAGGCGAGAGTCAAATATTCTCTGTAGACTATGAAA
AAAAGTAACAGATATCACGATCTAAGTGTTATCCCAATCCATTCATCATG
AGTTCCTTAAAGAAGATTCTCGGTCTGAAGGGGAAAGGTAAGAAATCTAA
GAAATTAGGGATCGCACCACCCCCTTATGAAGAGGACACTAGCATGGAGT
ATGCTCCGAGCGCTCCAATTGACAAATCCTATTTTGGAGTTGACGAGATG
GACACCTATGATCCGAATCAATTAAGATATGAGAAATTCTTCTTTACAGTG
AAAATGACGGTTAGATCTAATCGTCCGTTCAGAACATACTCAGATGTGGC
AGCCGCTGTATCCCATTGGGATCACATGTACATCGGAATGGCAGGGAAAC
GTCCCTTCTACAAAATCTTGGCTTTTTTGGGTTCTTCTAATCTAAAGGCCAC
TCCAGCGGTATTGGCAGATCAAGGTCAACCAGAGTATCACACTCACTGCG
AAGGCAGGGCTTATTTGCCACATAGGATGGGGAAGACCCCTCCCATGCTC
AATGTACCAGAGCACTTCAGAAGACCATTCAATATAGGTCTTTACAAGGG
AACGATTGAGCTCACAATGACCATCTACGATGATGAGTCACTGGAAGCAG
CTCCTATGATCTGGGATCATTTCAATTCTTCCAAATTTTCTGATTTCAGAGA
GAAGGCCTTAATGTTTGGCCTGATTGTCGAGAAAAAGGCATCTGGAGCGT
GGGTCCTGGATTCTATCAGCCACTTCAAATGAGCTAGTCTAACTTCTAGCT
TCTGAACAATCCCCGGTTTACTCAGTCTCTCCTAATTCCAGCCTCTCGAAC
AACTAATATCCTGTCTTTTCTATCCCTATGAAAAAAATTTTCATAGATTCA
ACTGTTTTCATAGTAAAACCAACGTAACTAAGCTTTTTCATAGATTCAACT
GTTTTCATAGTAAAACCAACGTAACTAAGCTCTAACAGAGATCGATCTGTT
TCCTTGACACTATGAAGTGCCTTTTGTACTTAGCCTTTTTATTCATTGGGGT
GAATTGCATGTTTGTTTTTCTTGTTTTATTGCCACTAGTCTCTAGTCAGTGT
GTTAATCTTACAACCAGAACTCAATTACCCCCTGCATACACTAATTCTTTC
ACACGTGGTGTTTATTACCCTGACAAAGTTTTCAGATCCTCAGTTTTACAT
TCAACTCAGGACTTGTTCTTACCTTTCTTTTCCAATGTTACTTGGTTCCATG
CTATACATGTCTCTGGGACCAATGGTACTAAGAGGTTTGATAACCCTGTCC
TACCATTTAATGATGGTGTTTATTTTGCTTCCACTGAGAAGTCTAACATAA
TAAGAGGCTGGATTTTTGGTACTACTTTAGATTCGAAGACCCAGTCCCTAC
TTATTGTTAATAACGCTACTAATGTTGTTATTAAAGTCTGTGAATTTCAATT
TTGTAATGATCCATTTTTGGGTGTTTATTACCACAAAAACAACAAAAGTTG
GATGGAAAGTGAGTTCAGAGTTTATTCTAGTGCGAATAATTGCACTTTTGA
ATATGTCTCTCAGCCTTTTCTTATGGACCTTGAAGGAAAACAGGGTAATTT
CAAAAATCTTAGGGAATTTGTGTTTAAGAATATTGATGGTTATTTTAAAAT
ATATTCTAAGCACACGCCTATTAATTTAGTGCGTGATCTCCCTCAGGGTTT
TTCGGCTTTAGAACCATTGGTAGATTTGCCAATAGGTATTAACATCACTAG
GTTTCAAACTTTACTTGCTTTACATAGAAGTTATTTGACTCCTGGTGATTCT
TCTTCAGGTTGGACAGCTGGTGCTGCAGCTTATTATGTGGGTTATCTTCAA
CCTAGGACTTTTCTATTAAAATATAATGAAAATGGAACCATTACAGATGCT
GTAGACTGTGCACTTGACCCTCTCTCAGAAACAAAGTGTACGTTGAAATC
CTTCACTGTAGAAAAAGGAATCTATCAAACTTCTAACTTTAGAGTCCAACC
AACAGAATCTATTGTTAGATTTCCTAATATTACAAACTTGTGCCCTTTTGG
TGAAGTTTTTAACGCCACCAGATTTGCATCTGTTTATGCTTGGAACAGGAA
GAGAATCAGCAACTGTGTTGCTGATTATTCTGTCCTATATAATTCCGCATC
ATTTTCCACTTTTAAGTGTTATGGAGTGTCTCCTACTAAATTAAATGATCTC
TGCTTTACTAATGTCTATGCAGATTCATTTGTAATTAGAGGTGATGAAGTC
AGACAAATCGCTCCAGGGCAAACTGGAAAGATTGCTGATTATAATTATAA
ATTACCAGATGATTTTACAGGCTGCGTTATAGCTTGGAATTCTAACAATCT
TGATTCTAAGGTTGGTGGTAATTATAATTACCTGTATAGATTGTTTAGGAA
GTCTAATCTCAAACCTTTTGAGAGAGATATTTCAACTGAAATCTATCAGGC
CGGTAGCACACCTTGTAATGGTGTTGAAGGTTTTAATTGTTACTTTCCTTT
ACAATCATATGGTTTCCAACCCACTAATGGTGTTGGTTACCAACCATACAG
AGTAGTAGTACTTTCTTTTGAACTTCTACATGCACCAGCAACTGTTTGTGG
ACCTAAAAAGTCTACTAATTTGGTTAAAAACAAATGTGTCAATTTCAACTT
CAATGGTTTAACAGGCACAGGTGTTCTTACTGAGTCTAACAAAAAGTTTCT
GCCTTTCCAACAATTTGGCAGAGACATTGCTGACACTACTGATGCTGTCCG
TGATCCACAGACACTTGAGATTCTTGACATTACACCATGTTCTTTTGGTGG
TGTCAGTGTTATAACACCAGGAACAAATACTTCTAACCAGGTTGCTGTTCT
TTATCAGGATGTTAACTGCACAGAAGTCCCTGTTGCTATTCATGCAGATCA
ACTTACTCCTACTTGGCGTGTTTATTCTACAGGTTCTAATGTTTTTCAAACA
CGTGCAGGCTGTTTAATAGGGGCTGAACATGTCAACAACTCATATGAGTG
TGACATACCCATTGGTGCAGGTATATGCGCTAGTTATCAGACTCAGACTA
ATTCTCCTCGGCGGGCACGTAGTGTAGCTAGTCAATCCATCATTGCCTACA
CTATGTCACTTGGTGCAGAAAATTCAGTTGCTTACTCTAATAACTCTATTG
CCATACCCACAAATTTTACTATTAGTGTTACCACAGAAATTCTACCAGTGT
CTATGACCAAGACATCAGTAGATTGTACAATGTACATTTGTGGTGATTCAA
CTGAATGCAGCAATCTTTTGTTGCAATATGGCAGTTTTTGTACACAATTAA
ACCGTGCTTTAACTGGAATAGCTGTTGAACAAGACAAAAACACCCAAGAA
GTTTTTGCACAAGTCAAACAAATTTACAAAACACCACCAATTAAAGATTTT
GGTGGTTTTAATTTTTCACAAATATTACCAGATCCATCAAAACCAAGCAAG
AGGTCATTTATTGAAGATCTACTTTTCAACAAAGTGACACTTGCAGATGCT
GGCTTCATCAAACAATATGGTGATTGCCTTGGTGATATTGCTGCTAGAGAC
CTCATTTGTGCACAAAAGTTTAACGGCCTTACTGTTTTGCCACCTTTGCTC
ACAGATGAAATGATTGCTCAATACACTTCTGCACTGTTAGCGGGTACAAT
CACTTCTGGTTGGACCTTTGGTGCAGGTGCTGCATTACAAATACCATTTGC
TATGCAAATGGCTTATAGGTTTAATGGTATTGGAGTTACACAGAATGTTCT
CTATGAGAACCAAAAATTGATTGCCAACCAATTTAATAGTGCTATTGGCA
AAATTCAAGACTCACTTTCTTCCACAGCAAGTGCACTTGGAAAACTTCAA
GATGTGGTCAACCAAAATGCACAAGCTTTAAACACGCTTGTTAAACAACT
TAGCTCCAATTTTGGTGCAATTTCAAGTGTTTTAAATGATATCCTTTCACGT
CTTGACAAAGTTGAGGCTGAAGTGCAAATTGATAGGTTGATCACAGGCAG
ACTTCAAAGTTTGCAGACATATGTGACTCAACAATTAATTAGAGCTGCAG
AAATCAGAGCTTCTGCTAATCTTGCTGCTACTAAAATGTCAGAGTGTGTAC
TTGGACAATCAAAAAGAGTTGATTTTTGTGGAAAGGGCTATCATCTTATGT
CCTTCCCTCAGTCAGCACCTCATGGTGTAGTCTTCTTGCATGTGACTTATGT
CCCTGCACAAGAAAAGAACTTCACAACTGCTCCTGCCATTTGTCATGATG
GAAAAGCACACTTTCCTCGTGAAGGTGTCTTTGTTTCAAATGGCACACACT
GGTTTGTAACACAAAGGAATTTTTATGAACCACAAATCATTACTACAGAC
AACACATTTGTGTCTGGTAACTGTGATGTTGTAATAGGAATTGTCAACAAC
ACAGTTTATGATCCTTTGCAACCTGAATTAGACTCATTCAAGGAGGAGTTA
GATAAATATTTTAAGAATCATACATCACCAGATGTTGATTTAGGTGACATC
TCTGGCATTAATGCTTCAGTTGTAAACATTCAAAAAGAAATTGACCGCCTC
AATGAGGTTGCCAAGAATTTAAATGAATCTCTCATCGATCTCCAAGAACTT
GGAAAGTATGAGCAGTATATAAAATGGCCATGGTACATTTGGCTAGGTTT
TATAGCTGGCTTGATTGCCATAGTAATGGTGACAATTATGCTTTGCTGTAT
GACCAGTTGCTGTAGTTGTCTCAAGGGCTGTTGTTCTTGTGGATCCTGCTG
CAAATTTGATGAAGACGACTCTGAGCCAGTGCTCAAAGGAGTCAAATTAC
ATTACACATAATGAAAAAAATCATCCCAATAGTGCTAATACTAATGCCGT
CAACTGTTTGCTATGTCTGATAATGGACCCCAAAATCAGCGAAATGCACC
CCGCATTACGTTTGGTGGACCCTCAGATTCAACTGGCAGTAACCAGAATG
GAGAACGCAGTGGGGCGCGATCAAAACAACGTCGGCCCCAAGGTTTACCC
AATAATACTGCGTCTTGGTTCACCGCTCTCACTCAACATGGCAAGGAAGA
CCTTAAATTCCCTCGAGGACAAGGCGTTCCAATTAACACCAATAGCAGTC
CAGATGACCAAATTGGCTACTACCGAAGAGCTACCAGACGAATTCGTGGT
GGTGACGGTAAAATGAAAGATCTCAGTCCAAGATGGTATTTCTACTACCT
AGGAACTGGGCCAGAAGCTGGACTTCCCTATGGTGCTAACAAAGACGGCA
TCATATGGGTTGCAACTGAGGGAGCCTTGAATACACCAAAAGATCACATT
GGCACCCGCAATCCTGCTAACAATGCTGCAATCGTGCTACAACTTCCTCAA
GGAACAACATTGCCAAAAGGCTTCTACGCAGAAGGGAGCAGAGGCGGCA
GTCAAGCCTCTTCTCGTTCCTCATCACGTAGTCGCAACAGTTCAAGAAATT
CAACTCCAGGCAGCAGTAGGGGAACTTCTCCTGCTAGAATGGCTGGCAAT
GGCGGTGATGCTGCTCTTGCTTTGCTGCTGCTTGACAGATTGAACCAGCTT
GAGAGCAAAATGTCTGGTAAAGGCCAACAACAACAAGGCCAAACTGTCA
CTAAGAAATCTGCTGCTGAGGCTTCTAAGAAGCCTCGGCAAAAACGTACT
GCCACTAAAGCATACAATGTAACACAAGCTTTCGGCAGACGTGGTCCAGA
ACAAACCCAAGGAAATTTTGGGGACCAGGAACTAATCAGACAAGGAACT
GATTACAAACATTGGCCGCAAATTGCACAATTTGCCCCCAGCGCTTCAGC
GTTCTTCGGAATGTCGCGCATTGGCATGGAAGTCACACCTTCGGGAACGT
GGTTGACCTACACAGGTGCCATCAAATTGGATGACAAAGATCCAAATTTC
AAAGATCAAGTCATTTTGCTGAATAAGCATATTGACGCATACAAAACATT
CCCACCAACAGAGCCTAAAAAGGACAAAAAGAAGAAGGCTGATGAAACT
CAAGCCTTACCGCAGAGACAGAAGAAACAGCAAACTGTGACTCTTCTTCC
TGCTGCAGATTTGGATGATTTCTCCAAACAATTGCAACAATCCATGAGCA
GTGCTGACTCAACTCAGGCCTAATTTTCCCTATAAATAGGCCGTCTAATAT
TTTAGTGCTTTTAACTAGCATAATAAACAATACAACGTTTGCTGTGCTACA
TAGGCCGTCTAGTCTAGCTAATTAACAGCAATCATGGAAGTCCACGATTTT
GAGACCGACGAGTTCAATGATTTCAATGAAGATGACTATGCCACAAGAGA
ATTCCTGAATCCCGATGAGCGCATGACGTACTTGAATCATGCTGATTACAA
TTTGAATTCTCCTCTAATTAGTGATGATATTGACAATTTGATCAGGAAATT
CAATTCTCTTCCGATTCCCTCGATGTGGGATAGTAAGAACTGGGATGGAGT
TCTTGAGATGTTAACATCATGTCAAGCCAATCCCATCTCAACATCTCAGAT
GCATAAATGGATGGGAAGTTGGTTAATGTCTGATAATCATGATGCCAGTC
AAGGGTATAGTTTTTTACATGAAGTGGACAAAGAGGCAGAAATAACATTT
GACGTGGTGGAGACCTTCATCCGCGGCTGGGGCAACAAACCAATTGAATA
CATCAAAAAGGAAAGATGGACTGACTCATTCAAAATTCTCGCTTATTTGT
GTCAAAAGTTTTTGGACTTACACAAGTTGACATTAATCTTAAATGCTGTCT
CTGAGGTGGAATTGCTCAACTTGGCGAGGACTTTCAAAGGCAAAGTCAGA
AGAAGTTCTCATGGAACGAACATATGCAGGATTAGGGTTCCCAGCTTGGG
TCCTACTTTTATTTCAGAAGGATGGGCTTACTTCAAGAAACTTGATATTCT
AATGGACCGAAACTTTCTGTTAATGGTCAAAGATGTGATTATAGGGAGGA
TGCAAACGGTGCTATCCATGGTATGTAGAATAGACAACCTGTTCTCAGAG
CAAGACATCTTCTCCCTTCTAAATATCTACAGAATTGGAGATAAAATTGTG
GAGAGGCAGGGAAATTTTTCTTATGACTTGATTAAAATGGTGGAACCGAT
ATGCAACTTGAAGCTGATGAAATTAGCAAGAGAATCAAGGCCTTTAGTCC
CACAATTCCCTCATTTTGAAAATCATATCAAGACTTCTGTTGATGAAGGGG
CAAAAATTGACCGAGGTATAAGATTCCTCCATGATCAGATAATGAGTGTG
AAAACAGTGGATCTCACACTGGTGATTTATGGATCGTTCAGACATTGGGG
TCATCCTTTTATAGATTATTACACTGGACTAGAAAAATTACATTCCCAAGT
AACCATGAAGAAAGATATTGATGTGTCATATGCAAAAGCACTTGCAAGTG
ATTTAGCTCGGATTGTTCTATTTCAACAGTTCAATGATCATAAAAAGTGGT
TCGTGAATGGAGACTTGCTCCCTCATGATCATCCCTTTAAAAGTCATGTTA
AAGAAAATACATGGCCCACAGCTGCTCAAGTTCAAGATTTTGGAGATAAA
TGGCATGAACTTCCGCTGATTAAATGTTTTGAAATACCCGACTTACTAGAC
CCATCGATAATATACTCTGACAAAAGTCATTCAATGAATAGGTCAGAGGT
GTTGAAACATGTCCGAATGAATCCGAACACTCCTATCCCTAGTAAAAAGG
TGTTGCAGACTATGTTGGACACAAAGGCTACCAATTGGAAAGAATTTCTT
AAAGAGATTGATGAGAAGGGCTTAGATGATGATGATCTAATTATTGGTCT
TAAAGGAAAGGAGAGGGAACTGAAGTTGGCAGGTAGATTTTTCTCCCTAA
TGTCTTGGAAATTGCGAGAATACTTTGTAATTACCGAATATTTGATAAAGA
CTCATTTCGTCCCTATGTTTAAAGGCCTGACAATGGCGGACGATCTAACTG
CAGTCATTAAAAAGATGTTAGATTCCTCATCCGGCCAAGGATTGAAGTCA
TATGAGGCAATTTGCATAGCCAATCACATTGATTACGAAAAATGGAATAA
CCACCAAAGGAAGTTATCAAACGGCCCAGTGTTCCGAGTTATGGGCCAGT
TCTTAGGTTATCCATCCTTAATCGAGAGAACTCATGAATTTTTTGAGAAAA
GTCTTATATACTACAATGGAAGACCAGACTTGATGCGTGTTCACAACAAC
ACACTGATCAATTCAACCTCCCAACGAGTTTGTTGGCAAGGACAAGAGGG
TGGACTGGAAGGTCTACGGCAAAAAGGATGGACTATCCTCAATCTACTGG
TTATTCAAAGAGAGGCTAAAATCAGAAACACTGCTGTCAAAGTCTTGGCA
CAAGGTGATAATCAAGTTATTTGCACACAGTATAAAACGAAGAAATCGAG
AAACGTTGTAGAATTACAGGGTGCTCTCAATCAAATGGTTTCTAATAATG
AGAAAATTATGACTGCAATCAAAATAGGGACAGGGAAGTTAGGACTTTTG
ATAAATGACGATGAGACTATGCAATCTGCAGATTACTTGAATTATGGAAA
AATACCGATTTTCCGTGGAGTGATTAGAGGGTTAGAGACCAAGAGATGGT
CACGAGTGACTTGTGTCACCAATGACCAAATACCCACTTGTGCTAATATA
ATGAGCTCAGTTTCCACAAATGCTCTCACCGTAGCTCATTTTGCTGAGAAC
CCAATCAATGCCATGATACAGTACAATTATTTTGGGACATTTGCTAGACTC
TTGTTGATGATGCATGATCCTGCTCTTCGTCAATCATTGTATGAAGTTCAA
GATAAGATACCGGGCTTGCACAGTTCTACTTTCAAATACGCCATGTTGTAT
TTGGACCCTTCCATTGGAGGAGTGTCGGGCATGTCTTTGTCCAGGTTTTTG
ATTAGAGCCTTCCCAGATCCCGTAACAGAAAGTCTCTCATTCTGGAGATTC
ATCCATGTACATGCTCGAAGTGAGCATCTGAAGGAGATGAGTGCAGTATT
TGGAAACCCCGAGATAGCCAAGTTTCGAATAACTCACATAGACAAGCTAG
TAGAAGATCCAACCTCTCTGAACATCGCTATGGGAATGAGTCCAGCGAAC
TTGTTAAAGACTGAGGTTAAAAAATGCTTAATCGAATCAAGACAAACCAT
CAGGAACCAGGTGATTAAGGATGCAACCATATATTTGTATCATGAAGAGG
ATCGGCTCAGAAGTTTCTTATGGTCAATAAATCCTCTGTTCCCTAGATTTTT
AAGTGAATTCAAATCAGGCACTTTTTTGGGAGTCGCAGACGGGCTCATCA
GTCTATTTCAAAATTCTCGTACTATTCGGAACTCCTTTAAGAAAAAGTATC
ATAGGGAATTGGATGATTTGATTGTGAGGAGTGAGGTATCCTCTTTGACA
CATTTAGGGAAACTTCATTTGAGAAGGGGATCATGTAAAATGTGGACATG
TTCAGCTACTCATGCTGACACATTAAGATACAAATCCTGGGGCCGTACAG
TTATTGGGACAACTGTACCCCATCCATTAGAAATGTTGGGTCCACAACATC
GAAAAGAGACTCCTTGTGCACCATGTAACACATCAGGGTTCAATTATGTTT
CTGTGCATTGTCCAGACGGGATCCATGACGTCTTTAGTTCACGGGGACCAT
TGCCTGCTTATCTAGGGTCTAAAACATCTGAATCTACATCTATTTTGCAGC
CTTGGGAAAGGGAAAGCAAAGTCCCACTGATTAAAAGAGCTACACGTCTT
AGAGATGCTATCTCTTGGTTTGTTGAACCCGACTCTAAACTAGCAATGACT
ATACTTTCTAACATCCACTCTTTAACAGGCGAAGAATGGACCAAAAGGCA
GCATGGGTTCAAAAGAACAGGGTCTGCCCTTCATAGGTTTTCGACATCTCG
GATGAGCCATGGTGGGTTCGCATCTCAGAGCACTGCAGCATTGACCAGGT
TGATGGCAACTACAGACACCATGAGGGATCTGGGAGATCAGAATTTCGAC
TTTTTATTCCAAGCAACGTTGCTCTATGCTCAAATTACCACCACTGTTGCA
AGAGACGGATGGATCACCAGTTGTACAGATCATTATCATATTGCCTGTAA
GTCCTGTTTGAGACCCATAGAAGAGATCACCCTGGACTCAAGTATGGACT
ACACGCCCCCAGATGTATCCCATGTGCTGAAGACATGGAGGAATGGGGAA
GGTTCGTGGGGACAAGAGATAAAACAGATCTATCCTTTAGAAGGGAATTG
GAAGAATTTAGCACCTGCTGAGCAATCCTATCAAGTCGGCAGATGTATAG
GTTTTCTATATGGAGACTTGGCGTATAGAAAATCTACTCATGCCGAGGAC
AGTTCTCTATTTCCTCTATCTATACAAGGTCGTATTAGAGGTCGAGGTTTC
TTAAAAGGGTTGCTAGACGGATTAATGAGAGCAAGTTGCTGCCAAGTAAT
ACACCGGAGAAGTCTGGCTCATTTGAAGAGGCCGGCCAACGCAGTGTACG
GAGGTTTGATTTACTTGATTGATAAATTGAGTGTATCACCTCCATTCCTTTC
TCTTACTAGATCAGGACCTATTAGAGACGAATTAGAAACGATTCCCCACA
AGATCCCAACCTCCTATCCGACAAGCAACCGTGATATGGGGGTGATTGTC
AGAAATTACTTCAAATACCAATGCCGTCTAATTGAAAAGGGAAAATACAG
ATCACATTATTCACAATTATGGTTATTCTCAGATGTCTTATCCATAGACTTC
ATTGGACCATTCTCTATTTCCACCACCCTCTTGCAAATCCTATACAAGCCA
TTTTTATCTGGGAAAGATAAGAATGAGTTGAGAGAGCTGGCAAATCTTTC
TTCATTGCTAAGATCAGGAGAGGGGTGGGAAGACATACATGTGAAATTCT
TCACCAAGGACATATTATTGTGTCCAGAGGAAATCAGACATGCTTGCAAG
TTCGGGATTGCTAAGGATAATAATAAAGACATGAGCTATCCCCCTTGGGG
AAGGGAATCCAGAGGGACAATTACAACAATCCCTGTTTATTATACGACCA
CCCCTTACCCAAAGATGCTAGAGATGCCTCCAAGAATCCAAAATCCCCTG
CTGTCCGGAATCAGGTTGGGCCAATTACCAACTGGCGCTCATTATAAAATT
CGGAGTATATTACATGGAATGGGAATCCATTACAGGGACTTCTTGAGTTG
TGGAGACGGCTCCGGAGGGATGACTGCTGCATTACTACGAGAAAATGTGC
ATAGCAGAGGAATATTCAATAGTCTGTTAGAATTATCAGGGTCAGTCATG
CGAGGCGCCTCTCCTGAGCCCCCCAGTGCCCTAGAAACTTTAGGAGGAGA
TAAATCGAGATGTGTAAATGGTGAAACATGTTGGGAATATCCATCTGACT
TATGTGACCCAAGGACTTGGGACTATTTCCTCCGACTCAAAGCAGGCTTG
GGGCTTCAAATTGATTTAATTGTAATGGATATGGAAGTTCGGGATTCTTCT
ACTAGCCTGAAAATTGAGACGAATGTTAGAAATTATGTGCACCGGATTTT
GGATGAGCAAGGAGTTTTAATCTACAAGACTTATGGAACATATATTTGTG
AGAGCGAAAAGAATGCAGTAACAATCCTTGGTCCCATGTTCAAGACGGTC
GACTTAGTTCAAACAGAATTTAGTAGTTCTCAAACGTCTGAAGTATATATG
GTATGTAAAGGTTTGAAGAAATTAATCGATGAACCCAATCCCGATTGGTC
TTCCATCAATGAATCCTGGAAAAACCTGTACGCATTCCAGTCATCAGAAC
AGGAATTTGCCAGAGCAAAGAAGGTTAGTACATACTTTACCTTGACAGGT
ATTCCCTCCCAATTCATTCCTGATCCTTTTGTAAACATTGAGACTATGCTAC
AAATATTCGGAGTACCCACGGGTGTGTCTCATGCGGCTGCCTTAAAATCAT
CTGATAGACCTGCAGATTTATTGACCATTAGCCTTTTTTATATGGCGATTA
TATCGTATTATAACATCAATCATATCAGAGTAGGACCGATACCTCCGAAC
CCCCCATCAGATGGAATTGCACAAAATGTGGGGATCGCTATAACTGGTAT
AAGCTTTTGGCTGAGTTTGATGGAGAAAGACATTCCACTATATCAACAGT
GTTTAGCAGTTATCCAGCAATCATTCCCGATTAGGTGGGAGGCTGTTTCAG
TAAAAGGAGGATACAAGCAGAAGTGGAGTACTAGAGGTGATGGGCTCCC
AAAAGATACCCGAACTTCAGACTCCTTGGCCCCAATCGGGAACTGGATCA
GATCTCTGGAATTGGTCCGAAACCAAGTTCGTCTAAATCCATTCAATGAG
ATCTTGTTCAATCAGCTATGTCGTACAGTGGATAATCATTTGAAATGGTCA
AATTTGCGAAGAAACACAGGAATGATTGAATGGATCAATAGACGAATTTC
AAAAGAAGACCGGTCTATACTGATGTTGAAGAGTGACCTACACGAGGAA
AACTCTTGGAGAGATTAAAAAATCATGAGGAGACTCCAAACTTTAAGTAT
G (SEQ ID NO: 3)

What is claimed is:

1. An immunogenic composition comprising a vesicular stomatitis virus (VSV), wherein the VSV expresses a spike glycoprotein of severe acute respiratory syndrome coronavirus 2 (SARS-CoV-2).

2. The composition of claim 1, wherein the VSV serotype is Indiana vesiculovirus or New Jersey vesiculovirus.

3. The composition of claim 1, wherein the VSV is modified to replace wildtype VSV glycoprotein with the spike glycoprotein of SARS-CoV-2 (COV-S).

4. The composition of claim 1, wherein the VSV is modified to incorporate a measles hemagglutinin gene.

5. The composition of claim 4, wherein the measles strain is Edmonston.

6. The composition of claim 4, wherein the hemagglutinin gene is inserted between the COV-S gene and the VSV-L gene.

7. The composition of claim 1, wherein the VSV is modified to incorporate the SARS-CoV-2 nucleocapsid phosphoprotein (COV-N).

8. The composition of claim 7, wherein COV-N is incorporated between the COV-S gene and the VSV-L gene.

9. The composition of claim 1, wherein the VSV is further modified to incorporate a genetic kill switch.

10. The composition of claim 10, wherein the genetic kill switch is inserted between the COV-S gene and the VSV-L gene.

11. The composition of claim 1, wherein the VSV is further modified to incorporate a reporter gene.

12. The composition of claim 11, wherein the reporter gene is selected from the group consisting of a fluorescent gene, a bioluminescent gene, and a gene related to clinical imaging modalities.

13. A method for preventing or treating an infection caused by a pathogen comprising administering to a subject an effective amount of an immunogenic composition comprising a vesicular stomatitis virus, wherein the VSV expresses a spike glycoprotein of severe acute respiratory syndrome coronavirus 2 (SARS-CoV-2).

14. The method of claim 13, wherein the VSV serotype is Indiana vesiculovirus or New Jersey vesiculovirus.

15. The method of claim 13, wherein the VSV is modified to replace wildtype VSV glycoprotein with the spike glycoprotein of SARS-CoV-2 (COV-S).

16. The method of claim 13, wherein the VSV is modified to incorporate a measles hemagglutinin gene (MV-H).

17. The method of claim 16, wherein the hemagglutinin gene is inserted between the COV-S gene and the VSV-L gene.

18. The method of claim 13, wherein the VSV is modified to incorporate the SARS-CoV-2 nucleocapsid phosphoprotein (COV-N).

19. The method of claim 19, wherein COV-N is incorporated between the COV-S gene and the VSV-L gene.

20. The method of claim 13, wherein the immunogenic composition is administered to the subject intramuscularly or via inhalation.


SEQUENCE LISTING (3 sequences)

SEQ ID NO: 1 - DNA, 13476 bp, Artificial
  vesicular stomatitis virus
acgaagacaaacaaaccattattatcattaaaaggctcaggagaaactttaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggtttgtatgcacaggatccagaagctgagcaagttgaaggctttatacaggggcctttagatgactatgcagatgaggaagtggatgttgtatttacttcggactggaaaccacctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacactcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctaacttctagcttctgaacaatccccggtttactcagtctctcctaattccagcctctcgaacaactaatatcctgtcttttctatccctatgaaaaaaattttcatagattcaactgttttcatagtaaaaccaacgtaactaagctctaacagagatcgatctgtttccttgacactatgaagtgccttttgtacttagcctttttattcattggggtgaattgcatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataatgaaaaaaatcatcccaatagtgctaatactaatgccgtcaactgtttgctgcataataaacaatacaacgtttgctgtgctacataggccgtctagtctagctaattaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggactatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaacttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatg

SEQ ID NO: 2 - DNA, 15511 bp, Artificial
  vesicular stomatitis virus
acgaagacaaacaaaccattattatcattaaaaggctcaggagaaactttaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggtttgtatgcacaggatccagaagctgagcaagttgaaggctttatacaggggcctttagatgactatgcagatgaggaagtggatgttgtatttacttcggactggaaaccacctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacactcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctaacttctagcttctgaacaatccccggtttactcagtctctcctaattccagcctctcgaacaactaatatcctgtcttttctatccctatgaaaaaaattttcatagattcaactgttttcatagtaaaaccaacgtaactaagctttttcatagattcaactgttttcatagtaaaaccaacgtaactaagctctaacagagatcgatctgtttccttgacactatgaagtgccttttgtacttagcctttttattcattggggtgaattgcatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataatgaaaaaaatcatcccaatagtgctaatactaatgccgtcaactgtttgctctaacagagatcgatctgtttccttgacactatgaagtgccttttgtacttagcctttttattcattggggtgaattgcatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagccagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcaatgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaggccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagtgaaaaaaattttccctataaataggccgtctaatattttagtgcttttaactagcataataaacaatacaacgtttgctgtgctacataggccgtctagtctagctaattaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggactatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaacttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatg

SEQ ID NO: 3 - DNA, 14829 bp, Artificial
  vesicular stomatitis virus
acgaagacaaacaaaccattattatcattaaaaggctcaggagaaactttaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggtttgtatgcacaggatccagaagctgagcaagttgaaggctttatacaggggcctttagatgactatgcagatgaggaagtggatgttgtatttacttcggactggaaaccacctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacactcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctaacttctagcttctgaacaatccccggtttactcagtctctcctaattccagcctctcgaacaactaatatcctgtcttttctatccctatgaaaaaaattttcatagattcaactgttttcatagtaaaaccaacgtaactaagctttttcatagattcaactgttttcatagtaaaaccaacgtaactaagctctaacagagatcgatctgtttccttgacactatgaagtgccttttgtacttagcctttttattcattggggtgaattgcatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataatgaaaaaaatcatcccaatagtgctaatactaatgccgtcaactgtttgctatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaattttccctataaataggccgtctaatattttagtgcttttaactagcataataaacaatacaacgtttgctgtgctacataggccgtctagtctagctaattaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggactatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaacttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatg